(19) United States
(12) Patent Application Publication
(10) Pub. No.: US 2022/0133758 A1
(43) Pub. Date: May 5, 2022

(54) TREATMENT OF INFLAMMATORY BOWEL DISEASES WITH 2'-FUCOSYLLACTOSE COMPOUNDS

(71) Applicant: CHILDREN'S HOSPITAL MEDICAL CENTER, Cincinnati, OH (US)

(72) Inventors: Ardythe L. MORROW, Cincinnati, OH (US); Lee A. DENSON, Cincinnati, OH (US); David S. NEWBURG, Newtonville, MA (US)

(73) Assignee: CHILDREN'S HOSPITAL MEDICAL CENTER, Cincinnati, OH (US)

(21) Appl. No.: 17/524,392

(22) Filed: Nov. 11, 2021

Related U.S. Application Data
(63) Continuation of application No. 16/500,935, filed on Oct. 4, 2019.
(60) Provisional application No. 62/482,840, filed on Apr. 7, 2017.

(51) Int. Cl. A61K 31/702; A61P 1/00; A61K 39/395

(52) U.S. Cl. CPC A61K 31/702; A61K 2039/505; A61K 39/3955; A61P 1/00

(57) ABSTRACT

Provided herein are 2′-fucosyllactose compounds and methods of using such for treating inflammatory bowel diseases (IBD) (e.g., Crohn's disease (CD) or ulcerative colitis (UC)) or alleviating or reducing the risk of relapse in IBD.

### CROSS-REFERENCE TO RELATED APPLICATIONS

This application claims the benefit under 35 U.S.C. § 119(e) of U.S. provisional application No. 62/482,840, filed Apr. 7, 2017, the contents of which are incorporated by reference herein in their entirety.

### BACKGROUND

The Inflammatory Bowel Diseases (IBDs), Crohn's Disease (CD) and Ulcerative Colitis (UC), are chronic and debilitating disorders with peak incidence in the second and third decades of life. While considerable progress has been made in optimizing anti-Tumor Necrosis Factor (TNF) therapy to induce remission in Crohn's Disease (CD) and Ulcerative Colitis (UC), relapse is common and often unpredictable. CD patients who experienced relapse after infliximab withdrawal to thiopurine therapy exhibited lower baseline levels of specific taxa including Bacteroides, Clostridium coccoides, and F. prausnitzii. Lower baseline levels of F. prausnitzii are also associated with higher relapse rates in UC. Suppression of mucosal inflammation with infliximab (monoclonal anti-TNF antibody) only partially corrects this dysbiosis. Accordingly, there is a need for alternative approach for stable maintenance of IBD remission.

### SUMMARY OF THE INVENTION

The present disclosure is, at least in part, based on the development of using a 2′-fucosyllactose compound such as 2′-fucosyllactose (2′-FL) to maintain remission in IBD patients. For example, 2′FL can be provided as a dietary supplement alone or as an adjunct to an immune suppression therapy such as an anti-inflammatory therapy.

Accordingly, one aspect of the present disclosures features a method for alleviating or reducing the risk of relapse in inflammatory bowel disease (IBD) by administering to a subject in need thereof an effective amount of a 2′-FL compound. For example, the subject can be a human IBD patient who has undergone or is on an anti-inflammatory therapy.

In another aspect, the present disclosure provides a method for treating IBD by administering to a subject in need thereof a 2′-FL compound in an amount equivalent to 1 mg/day to 20 mg/day of 2′-FL.

In any of the methods described herein, the subject in need of a 2′-FL compound can be a human patient at risk of developing IBD, suspected of having IBD, or having IBD. For example, the subject in need of a 2′-FL compound can be a human patient who is in remission of IBD (including, e.g., but not limited to Crohn's disease (CD) or ulcerative colitis (UC)), e.g., who has undergone or is receiving an anti-inflammatory therapy. The anti-inflammatory therapy can be an anti-TNF therapy such as use of TNF inhibitors including, e.g., but not limited to infliximab and/or adalimumab. The subject to be treated can be an adult or a children. The subject to be treated can be a FUT2 secretor or a FUT2 non-secretor. In some embodiments, the subject to be treated has a daily fiber intake of less than 7 g/1000 kcal. In some embodiments, the subject to be treated has a daily fiber intake of equal to or more than 7 g/1000 kcal. The subject to be treated may not be receiving a corticosteroid, an antibiotic, a probiotic, and/or a prebiotic that is not a 2′-FL compound.

The 2′-FL compound can be administered to the subjects in need thereof in an effective amount to achieve a desirable clinical effect. For example, a human IBD patient, e.g., who has undergone or is on an anti-inflammatory therapy and/or is in remission of the IBD, can be administered a 2′-FL compound in an amount sufficient to increase abundance of intestinal microbes that produce short-chain fatty acids (e.g., Bifidobacteria, Bacteroides, and/or Parabacteroides) in the human patient. As another example, a human IBD patient, e.g., who has undergone or is on an anti-inflammatory therapy and/or is in remission of the IBD, can be administered a 2′-FL compound in an amount sufficient to decrease intestinal calprotectin of the human patient. In some embodiments, the effective amount of the 2′-FL compound administered to a subject in need thereof may be equivalent to 1 mg/day to 20 mg/day of 2′-FL, 1 mg/day to 15 mg/day of 2′-FL, or 1 mg/day to 10 mg/day of 2′-FL.

The 2′-FL compound can be administered to the subject via any administration route, including, e.g., by oral administration. In some embodiments, the 2′-FL compound can be formulated as a pharmaceutical composition or a dietary supplement, e.g., suitable for oral administration. The composition can comprise the 2′-FL compound as the only oligosaccharide content or further comprises at least one additional oligosaccharide. An exemplary 2′-FL compound is 2′-FL. In some embodiments, a 2′-FL compound can be administered to a human patient who is receiving an anti-inflammatory as an adjuvant to the anti-inflammatory therapy.

Also within the scope of the present disclosure are (i) a pharmaceutical composition for use in treating IBD and/or alleviating or reducing the risk of relapse in IBD in a subject (e.g., as described herein), the composition comprising a 2′-FL compound as described herein and a pharmaceutically acceptable carrier; and (ii) use of a 2′-FL compound as described herein in manufacturing a medicament for use in treating IBD and/or alleviating or reducing the risk of relapse in IBD in a subject. The subject can be a human IBD patient who has undergone or is on an anti-inflammatory therapy, e.g., a human patient who is in remission of the IBD.

Also within the scope of the present disclosure are a dietary supplement for use in treating IBD and/or alleviating or reducing the risk of relapse in IBD in a subject, the composition comprising a 2′-FL compound as described herein. The subject can be a human IBD patient who has undergone or is on an anti-inflammatory therapy, e.g., a human patient is in remission of the IBD.

The details of one or more embodiments of the disclosure are set forth in the description below. Other features or advantages of the present disclosure will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 shows that microbial shifts and altered rectal mitochondrial gene expression in ulcerative colitis (UC) are addressed by 2′-FL supplementation in mice. 254 rectal biopsies and 293 stool samples were collected from 371 treatment-naive pediatric patients with UC at initial diagnosis. Samples were subjected to 16S rRNA amplicon sequencing and data were analyzed to infer the microbial taxonomic composition. The rectal global pattern of gene expression prior to therapy was determined using RNASeq in 206 UC patients and 18 healthy controls. The small bowel global pattern of gene expression was determined using RNASeq in mice with and without 2′-FL supplementation following ileocecal resection. Gene set enrichment analysis identified associated biologic processes. Gene signatures for mitochondrial biogenesis in treatment naïve pediatric UC and following 2′-FL supplementation in mice are also shown in the table as shown in FIG. 1.

FIG. 2 is a schematic diagram showing combined 2′-FL/anti-TNF therapy to increase microbial butyrate production and cellular butyrate responsiveness.

FIGS. 3A-3C are bar graphs showing taxa associated with newly diagnosed Crohn's disease (CD) and B2 Stricturing or B3 internal penetrating disease complications compared to B1 inflammatory behavior.

FIG. 4 is a bar graph showing differentially abundant taxa associated with disease severity in newly diagnosed ulcerative colitis.

FIG. 5 is a graph showing ileal gene signatures associated with disease complications in pediatric Crohn's disease.

FIG. 6 is a schematic diagram showing an overall patient stratification strategy for a clinical trial involving use of 2′-FL as a dietary supplement in pediatric and young adult IBD patients receiving stable maintenance anti-TNF therapy (left) and the allocation of doses at different stages of the clinical trial (right).

FIG. 7 is a schematic diagram showing an overall study design of a clinical trial that provides a pilot and feasibility study of 2′-FL as a dietary supplement in pediatric and young adult IBD patients receiving stable maintenance anti-TNF therapy.

FIG. 8 is a table showing a schedule of activities (SOA), which outlines study procedures and assessments performed at each visit during the clinical trial as shown in FIG. 7.

### DETAILED DESCRIPTION OF THE INVENTION

Inflammatory bowel diseases (IBDs) (including, e.g., ulcerative colitis and Crohn's disease) are chronic and debilitating disorders with peak incidence in the second and third decades of life. While considerable progress has been made in optimizing medications such as therapies targeting inflammatory cytokines to achieve remission in IBD, relapse is common and unpredictable. For example, it was reported that 37% of IBD patients (from a single treatment center) receiving infliximab or adalimumab anti-TNF therapy relapsed despite optimal therapeutic drug monitoring and dosing. In addition to adverse effects upon quality of life and work productivity, IBD relapses increase cost of care. Accordingly, there is a need to develop alternative methods and compositions for treatment of IBD as well as for maintenance of IBD remission to reduce the risk of IBD relapses.

The present disclosure is, at least in part, based on the development of using 2′-fucosyllactose (2′-FL) compound to maintain remission in IBD patients and thus to reduce the risk of relapse in IBD. For example, 2′-FL can be provided as a dietary supplement or in a pharmaceutical composition, either alone or as an adjunct to an immune suppression therapy such as an anti-inflammatory therapy. Administration of a 2′-FL compound (e.g., 2′FL) can increase microbial production of butyrate, which is an essential regulator of intestinal epithelial cell function. While treatment of IBD with anti-inflammatory therapy (e.g., anti-TNF inhibitors) suppresses intestinal inflammation to promote cellular responsiveness of intestinal endothelial cells to butyrate, it does not increase abundance of beneficial intestinal bacteria. Therefore, a combined 2′-FL/anti-TNF therapy can provide direct modulation of beneficial microbiota to increase microbial butyrate production while promoting cellular butyrate responsiveness by an anti-TNF therapy, thereby enhancing sustained clinical remission in IBD.

Accordingly, described herein are methods and compositions for treating IBD in subjects using a 2′-FL compound. When applied to human IBD patients who have undergone or are on an anti-inflammatory therapy (e.g., human patients who are in remission of the IBD), the treatment methods described herein are expected to alleviate or reduce the risk of relapse in IBD. Further, the treatment methods described herein are expected to be particularly effective to sustain IBD remission when a 2′-FL compound is provided as an adjuvant to an anti-inflammatory therapy (e.g., an anti-TNF therapy) being concurrently administered to patients who are in remission of the IBD.

In some aspects, the disclosure relates to methods for treating IBD or alleviating or reducing the risk of relapse in IBD using a 2′-FL compound, which can be provided as a dietary supplement or in a pharmaceutical composition. Such a dietary supplement or pharmaceutical composition can be used alone or as an adjunct to an anti-inflammatory therapy for IBD in subjects in need of the treatment.

### I. 2′-Fucosyllactose (2′-FL) Compounds and Compositions Comprising the Same

A 2-fucosyllactose (2′-FL) compound is an oligosaccharide having the three sugar units backbone as in the 2′-fucosyllactose (Fucα1, 2Galβ1,4Glc), wherein each of the sugar units (fucose (Fuc), galactose (Gal), and glucose (Glc)) can be independently either in its native form or in a modified form. For example, the modified form of a sugar unit can be a sugar unit, in which at least one or more (e.g., 1, 2, 3, or more) of the hydroxyl groups is replaced with a hydrogen, methyl, ethyl, or amine group.

In some embodiments, a 2′-FL compound is 2′-FL having a chemical structure of (Fucα1, 2Galβ1,4Glc), e.g., which is identical to the chemical structure of a native 2′-FL that is found in milk (e.g., human milk).

In some embodiments, a 2′-FL compound is a modified 2′-FL that retains at least 70% or more (including, e.g., at least 80%, at least 90%, at least 95%, at least 98%, at least 99% and up to 100%) of the biological functions of a native 2′-FL, e.g., the 2′-FL found in milk (e.g., human milk). In some embodiments, the modified 2′-FL can provide enhanced biological functions relative to that of a native 2′-FL, e.g., the 2′-FL found in milk (e.g., human milk). Such biological functions of 2′-FL include its beneficial effects on intestines, e.g., but are not limited to, anti-inflammatory effects, anti-bacterial adhesion effects, prebiotic effects (e.g., increasing abundance of beneficial microbiota such as short-chain fatty acid-producing microbes or butyrate-producing microbes). For example, in some embodiments, a modified 2′-FL is 2′-FL with (i) at least one or more of its hydroxyl groups to be replaced with a hydrogen, methyl, ethyl, or amine group, and/or (ii) the glucose at its reducing end to be replaced with N-acetylglucosamine.

The 2′-FL compounds described herein can be prepared by any methods known in the art. For example, the 2′-FL compounds can be synthesized chemically, purified from milk or produced in microorganisms. In some embodiments, the 2′-FL compounds described herein can be isolated from milk (e.g., human milk). For example, milk is first defatted by centrifugation to produce skimmed milk. The skimmed milk is then mixed with an organic solvent, such as acetone (e.g., 50% aqueous acetone) and ethanol (e.g., 67% aqueous ethanol), to precipitate milk proteins. Upon centrifugation, the supernatant is collected and subjected to chromatography. Oligosaccharide-containing fractions are collected and pooled. If necessary, the oligosaccharides thus prepared can be concentrated by conventional methods, e.g., dialysis or freeze-drying. Alternatively, 2′-FL compounds can also be isolated from skimmed milk by passing the skimmed milk through a 30,000 MWCO ultrafiltration membrane, collecting the diffusate, passing the diffusate through a 500 MWCO ultrafilter, and collecting the retentate, which contains milk oligosaccharides.

In some embodiments, the 2′-FL compounds described herein can be synthesized chemically or produced in microorganisms (e.g., by fermentation of recombinant microorganisms such as Escherichia coli, yeast, and Corynebacterium glutamicum). See, e.g., WO 2017/134176, WO 2016/153300, WO 2014/009921, WO 2010/115934, WO2005/055944, and U.S. Pat. No. 8,652,808, the relevant disclosures of which are incorporated by reference for the purposes or subject matter referenced herein.

In some embodiments, the 2′-FL compounds described herein can be provided in glycoconjugate form (e.g., glycoconjugates). As used herein, “glycoconjugates” refers to conjugates containing a sugar moiety (e.g., 2′-FL compounds) linked to another chemical species such as proteins, peptides, lipids, nucleic acids, and saccharides (e.g., oligosaccharides or polysaccharides). The 2′-FL compounds can be linked to other chemical species via a covalent or noncovalent bond, or via other forms of association, such as entrapment (e.g., of one moiety on or within the other, or of either or both entities on or within a third moiety). The glycoconjugates described herein can contain one or more (e.g., 1, 2, 3, or more) 2′-FL compounds linked to a chemical species such as a protein, a peptide, a lipid, a nucleic acid, or a saccharide. In one example, a 2′-FL compound is covalently linked via its reducing end sugar unit to a protein, a peptide, a lipid, a nucleic acid, or a saccharide (e.g., an oligosaccharide or a polysaccharide). For example, the reducing end sugar unit may be N-acetylglucosamine. In some embodiments where a 2′FL compound is linked to a saccharide (e.g., an oligosaccharide or a polysaccharide), the glycoconjugate is not a naturally occurring molecule that is found in milk. In some embodiments, the 2′-FL compounds in glycoconjugate form are not naturally occurring molecules.

Peptide backbones suitable for making the glycoconjugates described above include those having multiple glycosylation sites (e.g., asparagine, lysine, serine, or threonine residue) and low allergenic potential. Examples include, but are not limited to, amylase, bile salt-stimulated lipase, casein, folate-binding protein, globulin, gluten, haptocorrin, lactalbumin, lactoferrin, lactoperoxidase, lipoprotein lipase, lysozyme, mucin, ovalbumin, and serum albumin.

Typically, a 2′-FL compound can be covalently attached to a serine or threonine residue via an O-linkage or attached to an asparagine residue via an N-linkage. To form these linkages, the sugar unit at the reducing end of the 2′-FL compound is preferably an acetylated sugar unit, e.g., N-acetylgalactosamine, N-acetylglucosamine, and N-acetylmannosamine. A 2′-FL compound can be attached to a peptide (e.g., a protein) using standard methods. See, e.g., McBroom et al., Complex Carbohydrates, Part B, 28:212-219, 1972; Yariv et al., Biochem J., 85:383-388, 1962; Rosenfeld et al., Carbohydr. Res., 46:155-158, 1976; and Pazur, Adv. Carbohydr. Chem, Biochem., 39:405-447, 1981.

In one example, a 2′-FL compound is linked to a backbone molecule via a linker. Exemplary linkers are described in WO2005/055944. The 2′-FL compound can be bonded to a linker by an enzymatic reaction, e.g., a glycosyltransferase reaction. A number of glycosyltransferases, including fucosyltransferases, galactosyltransferases, glucosyltransferases, mannosyltransferases, galactosaminyltransferases, sialyltransferases and N-acetylglucosaminyltransferases, can be used to make the glycoconjugates described herein. More details about these glycosyltransferases can be found in U.S. Pat. Nos. 6,291,219; 6,270,987; 6,238,894; 6,204,431; 6,143,868; 6,087,143; 6,054,309; 6,027,928; 6,025,174; 6,025,173; 5,955,282; 5,945,322; 5,922,540; 5,892,070; 5,876,714; 5,874,261; 5,871,983; 5,861,293; 5,859,334; 5,858,752; 5,856,159; and 5,545,553.

Alternatively, the glycoconjugates described herein can be purified from milk by conventional methods e.g., by passing through ultrafiltration membranes, by precipitation in non-polar solvents, or through partition between immiscible solvents.

The 2′-FL compounds (either as a free oligosaccharide or in glycoconjugate form as described herein) may be formulated with one or more pharmaceutically acceptable carrier, diluent, and/or excipient to form a pharmaceutical composition. A carrier, diluent or excipient that is “pharmaceutically acceptable” includes one that is sterile and pyrogen free. Suitable pharmaceutical carriers, diluents and excipients are well known in the art. The carrier(s) must be “acceptable” in the sense of being compatible with the inhibitor and not deleterious to the recipients thereof.

A pharmaceutical composition comprising a 2′-FL compound (either as a free oligosaccharide or in glycoconjugate form as described herein) can be formulated according to routes of administration, including, e.g., parenteral administration, oral administration, buccal administration, sublingual administration, and topical administration.

In some embodiments, the pharmaceutical composition or formulation is suitable for oral, buccal or sublingual administration, such as in the form of powder, tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the 2′-FL compounds (either as a free oligosaccharide or in glycoconjugate form as described herein) may be combined with various sweetening or flavoring agents, coloring matter or dyes, with emulsifying and/or suspending agents and with diluents such as water, ethanol, propylene glycol and glycerin, and combinations thereof. For powder, the 2′-FL compounds (either as a free oligosaccharide or in glycoconjugate form as described herein) may be combined with various sweetening or flavoring agents, coloring matter or dyes, with emulsifying and/or suspending agents and with diluents such as water, ethanol, propylene glycol and glycerin, and combinations thereof.

In some embodiments, the pharmaceutical compositions or formulations are for parenteral administration, such as intravenous, intra-arterial, intra-muscular, subcutaneous, or intraperitoneal administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

Alternatively, the 2′-FL compounds (either as free oligosaccharides or in glycoconjugate form as described herein) can also be formulated as dietary supplements following methods well known in the food/dietary supplement industry. In one example, the dietary supplements comprising the 2′-FL compounds can be taken alone. In another example, the dietary supplements comprising the 2′-FL compounds can be incorporated into food products and/or beverages. Such food products and/or beverages may include, but not limited to, milk, milk formulas, yoghurt, cheese, ice-cream, cereals, among others. Such food products and/or beverages include also oral food supplements, nutritional drinks, and enteral nutrition preparation, for example for tube feeding administration.

In some embodiments, the formulations of any aspects described herein may comprise a 2′-fucosyllactose compound (either as a free oligosaccharide or in glycoconjugate form as described herein) as the only oligosaccharide content. In some embodiments, the formulations of any aspects described herein may comprise a 2′-fucosyllactose compound (either as a free oligosaccharide or in glycoconjugate form as described herein) as the only oligosaccharide content that provides a prebiotic effect. In some embodiments, the formulations of any aspects described herein may comprise a 2′-fucosyllactose compound (either as a free oligosaccharide or in glycoconjugate form as described herein) as the only oligosaccharide content that increases short-chain fatty acid-producing microbes in intestines, and/or increase microbial production of short-chain fatty acids (e.g., butyrate).

In alternative embodiments, the formulations of any aspects described herein may further comprise at least one or more additional (e.g., 1, 2, 3, or more) oligosaccharides. Examples of such oligosaccharides includes, but are not limited to other non-2′-FL milk saccharides, e.g., as shown in Tables 1-4 below, fructooligosaccharides (FOS), galacto-oligosaccharides (GOS), and any combinations thereof.

TABLE 1
Other fucosyl oligosaccharides

| | | |
|---|---|---|
| LNF-I | Lacto-N-fucopentaose | Fucα1,2Galβ1,3GlcNAcβ1,3Galβ1,4Glc |
| | I | |
| LNF-II | Lacto-N-fucopentaose II | 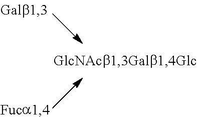 |
| 3′FL | 3-Fucosyllactose | 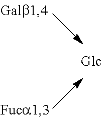 |
| LNF-III | Lacto-N-fucopentaose III | 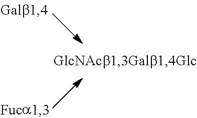 |
| LDFH-I | Lacto-N- difucohexaose I | 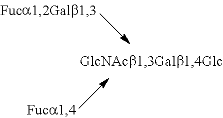 |
| LDFT | Lactodifucotetraose | 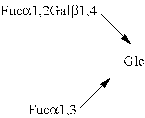 |

TABLE 2
Nonfucosylated, nonsialylated oligosaccharides

| | | |
|---|---|---|
| LNT | Lacto-N-tetraose | Galβ1,3GlcNAcβ1,3Galβ1,4Glc |
| LNneoT | Lacto-N-neotetraose | Galβ1,4GlcNAcβ1,3Galβ1,4Glc |

TABLE 3
Sialyl milk oligosaccharide structures

| | | |
|---|---|---|
| 3′-SL | 3′-Sialyllactose | NANAα2,3Galβ1,4Glc |
| 6′-SL | 6′-Sialyllactose | NANAα2,6Galβ1,4Glc |
| SLNT-c | Sialyllacto-N-neotetraose c | NANAα2,6Galβ1,4GlcNAcβ1,3Galβ1,4Glc |
| MSLNH | Monosialyllacto-N-hexaose | 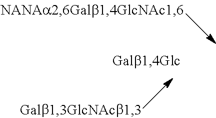 |
| DSLNH-I | Disialyllacto-N-hexaose I | 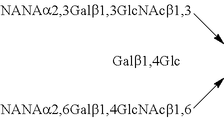 |
| MSLNnH-I | Monosialyllacto-N-neohexaose I | 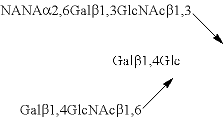 |
| SLNnH-II | Monosialyllacto-N-neohexaose II | 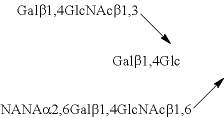 |
| DSLNnH | Disialyllacto-N-neohexaose | 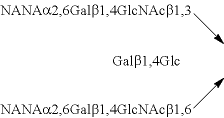 |
| DSLNT | Disialyllacto-N-tetraose | 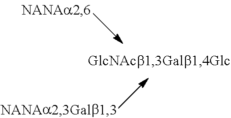 |
| DSLNH-II | Disialyllacto-N-hexaose II | 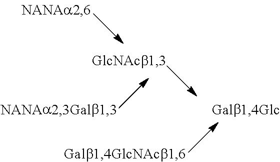 |
| SLNT-a | Sialyllacto-N-tetraose a | NANAα2,3Galβ1,3GlcNAcβ1,3 Galβ1,4Glc |
| DSLNH-I | Disialyllacto-N-hexaose I | 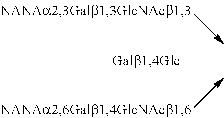 |
| SLNT-b | Sialyllacto-N-tetraose b | 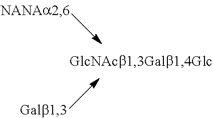 |

TABLE 4
Sialyl fucosyl oligosaccharides

| | | |
|---|---|---|
| 3′-S-3FL | 3′-Sialyl-3-fucosyllactose | 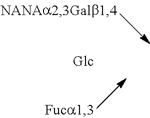 |
| DSFLNH | Disialomonofucosyllacto-N-neohexaose | 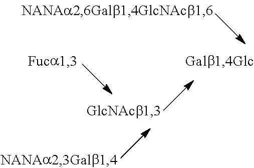 |
| MFMSLNO | Monofucosylmonosialyllacto-N-octaose (sialyl Lea) | 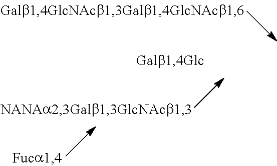 |
| SLNFH-II | Sialyllacto-N-fucohexaose II | 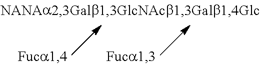 |
| DSLNFP-II | Disialyllacto-N-fucopentaose II | 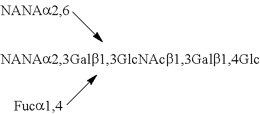 |
| MFDLNT | Monofucosyldisialyllacto-N-tetraose | 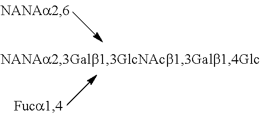 |

The formulations of any aspects described herein may be presented in unit-dose or multi-dose containers, for example sealed ampoules or vials, and may be stored in a freeze-dried (lyophilized) condition requiring only the addition of the sterile liquid carrier immediately prior to use.

### II. Subjects

In some embodiments, a subject to be treated by any of the methods described herein can be a mammal, e.g., a human, having, suspected of having, or at risk of developing an inflammatory bowel disease (IBD). IBDs are disorders that involve chronic inflammation of a digestive tract. Examples of IBDs include, but are not limited Crohn's disease (CD) and ulcerative colitis (UC).

IBD symptoms may vary, depending on the severity of inflammation and where it occurs. Symptoms may range from mild to severe. In some instances, IBD patients may experience periods of active illness followed by periods of remission. Signs and symptoms that are common to both Crohn's disease and ulcerative colitis include, but are not limited to diarrhea, fever and fatigue, abdominal pain and cramping, blood in your stool, reduced appetite, unintended weight loss, and any combinations thereof. Diagnosis for IBD are known in the art and can be determined by skilled practitioners, e.g., via blood test, various endoscopic procedures and/or imaging procedures.

In some embodiments, subjects to be treated by the methods described herein can be IBD subjects (e.g., human IBD patients) who have undergone or are on an anti-inflammatory and/or immune system suppression therapy. In some embodiments, subjects to be treated by the methods described herein are receiving an anti-inflammatory and/or immune system suppression therapy. An exemplary therapy of such includes, but is not limited to an anti-TNF therapy. Non-limiting examples of an anti-TNF therapy include infliximab, adalimumab, golimumab, natalizumab, vedolizumab, and ustekinumab. In some embodiments, subjects to be treated by the methods described herein are receiving an anti-TNF therapy comprising infliximab and/or adalimumab.

In some embodiments, subjects to be treated by the methods described herein can be IBD subjects (e.g., human IBD patients) who are in remission of the IBD. As used herein, the term “remission” refers to the disappearance or lessening of at least one or more symptoms associated with IBD, e.g., the ones described herein. Remission can be a complete remission (e.g., all signs or symptoms associated with IBD disappear) or a partial remission (e.g., at least one sign or symptom associated with IBD disappears or lessens).

In some embodiments, subjects to be treated by the methods described herein can be IBD subjects (e.g., human IBD patients) who are in remission of Crohn's disease (CD). For example, subjects are determined to be in remission of CD when they have a Crohn's disease activity index (CDAI) score of less than 150 (see, e.g., Merck Manuals; Best et al. “Development of a Crohn's disease activity index” Gastroenterology (1976) 70:439-444; and Best “Predicting the Crohn's disease activity index from the Harvey-Bradshaw Index” Inflamm Bowel Dis. (2006)12:304-310). In some embodiments, subjects are determined to be in remission of CD when they have a weighted pediatric Crohn's disease activity index (wPCDAI) score of less than 10 (see, e.g., Turner et al. “Which PCDAI Version Best Reflects Intestinal Inflammation in Pediatric Crohn Disease?” J Pediatr Gastroenterol Nutr (2017) 64:254-260).

In some embodiments, subjects to be treated by the methods described herein can be IBD subjects (e.g., human IBD patients) who are in remission of ulcerative colitis (UC). For example, subjects are determined to be in remission of UC according to any one of the disease activity index provided in Travis et al. “Review article: defining remission in ulcerative colitis” Aliment. Pharmacol. Ther. (2011) 34: 113-124. In some embodiments, subjects are determined to be in remission of UC when they have a modified Ulcerative Colitis Disease Activity Index (UCDAI) score less than or equal to 1, a UCDAI score less than or equal to 2, a Clinical Activity Index score less than or equal to 4, or a Mayo Clinic score less than or equal to 2 (with no subscore greater than 1). In some embodiments, subjects are determined to be in remission of UC when the subjects have complete cessation of rectal bleeding, urgency, and increased stool frequency, e.g., confirmed by endoscopic appearance of mucosal healing. See, e.g., Walsh and Travis “Assessing Disease Activity in Patients with Ulcerative Colitis” Gastroenterol Hepatol (NY) (2012) 8: 751-754. In some embodiments, subjects are determined to be in remission of UC when they have a pediatric ulcerative colitis activity index (PUCAI) score of less than 10 (see, e.g., Turner et al. “Appraisal of the pediatric ulcerative colitis activity index (PUCAI)” Inflamm Bower Dis (2009); 15:1218-23). In some embodiments, subjects to be treated by the methods described herein have a daily fiber intake of less than 7 g/1000 kcal. In some embodiments, subjects to be treated by the methods described herein have a daily fiber intake of equal to or more than 7 g/1000 kcal. The daily fiber intake can be determined, e.g., using Nutrition Data Systems for Research (NDSR) (Nutrition Coordinating Center, University of Minnesota, Minneapolis, Minn.) software and foods database to assess fiber intake. See, e.g., Sievert et al. “Maintenance of a nutrient database for clinical trials.” Control Clin Trials (1989)10:416-25.

In some embodiments, subjects to be treated by the methods described herein are not receiving a corticosteroid or an antibiotic that was indicated for treatment of IBD.

In some embodiments, subjects to be treated by the methods described herein can be a FUT2 secretor. In some embodiments, subjects to be treated by the methods described herein can be a FUT2 non-secretor. FUT2 corresponds to fucosyltransferase 2 gene, which is involved in the production of 2′-FL. Individuals with an inactivating polymorphism in the FUT2 gene are FUT2 non-secretors. FUT2 non-secretors are deficient in innate gut carbohydrates containing fucose, which increases susceptibility to microbial dysregulation and chronic inflammation.

Subjects to be treated by the methods described herein can be of any age. In some embodiments, a subject to be treated by the methods described herein can be a child, for example, a subject who is 18 years old or younger, e.g., 6 months-18 years old, inclusive. In some embodiments, the subject may be a child at the age of 11 or over, e.g., 11-18 years old, inclusive. In some embodiments, the subject may be a child at the age of 5-10. In some embodiments, the subject may be a child under the age of 5, e.g., 6 months to 4 years old, inclusive.

In some embodiments, a subject to be treated by the methods described herein can be an adult who is over the age of 18, such as 19-80 years old, inclusive. In some embodiments, an adult subject is at the age of 19-25. In some embodiments, an adult subject to be treated by the methods described herein may be above 25 (e.g., 25-80 years old, inclusive). In some embodiments, an adult subject to be treated by the methods described herein may be an elderly who is over the age of 65, such as 66-80 years old.

In some embodiments, subjects to be treated by the methods described herein may be at the age of 11 to 25.

A subject who needs the treatment as described herein can be identified via routine medical examination.

### III. Treatment of Inflammatory Bowel Diseases (IBDs)

Any of the 2′-FL compounds (either as a free oligosaccharide or in glycoconjugate form as described herein) and/or compositions comprising the same, e.g., those described herein, can be administered to a subject in need thereof, e.g., those described herein, for treating IBD, e.g., Crohn's disease (CD) or ulcerative colitis (UC). For example, in some embodiments, the subject is a human patient at risk of developing IBD, e.g., CD or UC. In some embodiments, the subject is a human patient having IBD, e.g., CD or UC. In some embodiments, the subject is a human IBD patient who has undergone or is on an immune system suppression and/or anti-inflammatory therapy (e.g., an anti-TNF therapy). In some embodiments, the subject is a human IBD patient who is in remission of the IBD and is receiving an immune system suppression and/or anti-inflammatory therapy (e.g., an anti-TNF therapy).

Any of the 2′-FL compounds and/or compositions comprising the same, e.g., those described herein, can be administered to a subject of any age who is in need of IBD treatment. In some embodiments, a subject to be administered a 2′-FL compound and/or composition described herein can be a child, for example, a subject who is 18 years old or younger, e.g., 6 months-18 years old, inclusive. In some embodiments, the subject may be a child at the age of 11 or over, e.g., 11-18 years old, inclusive. In some embodiments, the subject may be a child at the age of 5-10. In some embodiments, the subject may be a child under the age of 5, e.g., 6 months to 4 years old, inclusive.

In some embodiments, a subject to be administered a 2′-FL compound and/or composition described herein can be an adult who is over the age of 18, such as 19-80 years old, inclusive. In some embodiments, an adult subject is at the age of 19-25. In some embodiments, an adult subject to be administered a 2′-FL compound and/or composition described herein may be above 25 (e.g., 25-80 years old, inclusive). In some embodiments, an adult subject to be administered a 2′-FL compound and/or composition described herein may be an elderly who is over the age of 65, such as 66-80 years old.

In some embodiments, subjects to be administered a 2′-FL compound and/or composition described herein may be at the age of 11 to 25.

The term “treating” or “treatment” as used herein refers to application or administration of a 2′-FL compound (e.g., ones described herein, either as a free oligosaccharide or in glycoconjugate form as described herein), as a monotherapy or as a combined treatment (e.g., as an adjunct to an immune system suppression and/or anti-inflammatory therapy) to a subject, who has IBD, a symptom of IBD, or a predisposition toward IBD, with the purpose to cure, heal, alleviate, relieve, alter, remedy, ameliorate, improve, or affect the disease, the symptoms of the disease, or the predisposition toward the disease. The term “treating” or “treatment” also includes application or administration of a 2′-FL compound (e.g., ones described herein, either as a free oligosaccharide or in glycoconjugate form as described herein), as a monotherapy or as a combined treatment (e.g., as an adjunct to an immune system suppression and/or anti-inflammatory therapy) to a subject who is in remission of IBD, with the purpose to maintain the remission and thus alleviate, reduce, prevent, or delay the relapse occurrence.

In some embodiments, the treatment is prophylactic. The term “prophylactic” refers to application or administration of a 2′-FL compound (e.g., ones described herein, either as a free oligosaccharide or in glycoconjugate form as described herein), as a monotherapy or as a combined treatment (e.g., as an adjunct to an immune system suppression and/or anti-inflammatory therapy) to a subject who is at risk for IBD that prevents the occurrence, or delays the onset, of IBD.

In some embodiments, the treatment is therapeutic. The term “therapeutic” refers to application or administration of a 2′-FL compound (e.g., ones described herein, either as a free oligosaccharide or in glycoconjugate form as described herein), as a monotherapy or as a combined treatment (e.g., as an adjunct to an immune system suppression and/or anti-inflammatory therapy) to a subject, who has IBD or a symptom of IBD that improves at least one or more symptoms associated with IBD, e.g., reduced diarrhea, reduced blood in stool, and/or reduced frequency of symptom relapse.

For example, a treatment is therapeutic when application or administration of a 2′-FL compound (e.g., ones described herein, either as a free oligosaccharide or in glycoconjugate form as described herein), as a monotherapy or as a combined treatment (e.g., as an adjunct to an immune system suppression and/or anti-inflammatory therapy) alleviates or reduces the risk of IBD symptom relapse. As used herein, the term “relapse” refers to the occurrence or worsening of at least one or more symptoms associated with IBD.

In some embodiments, the treatment is therapeutic when application or administration of a 2′-FL compound (e.g., ones described herein), as a monotherapy or as a combined treatment (e.g., as an adjunct to an immune system suppression and/or anti-inflammatory therapy) alleviates or reduces the risk of symptom relapse in Crohn's disease (CD). For example, a human patient is determined to have a CD relapse when the Crohn's disease activity index (CDAI) score is increased to 150 or greater. In some embodiments, a human patient is determined to have a CD relapse when there is an increase of 20 or more points for the wPCDAI, e.g., between the start of the treatment and 4 weeks after.

In some embodiments, the treatment is therapeutic when application or administration of a 2′-FL compound (e.g., ones described herein), as a monotherapy or as a combined treatment (e.g., as an adjunct to an immune system suppression and/or anti-inflammatory therapy) alleviates or reduces the risk of symptom relapse in ulcerative colitis (UC). For example, a human patient is determined to have a UC relapse when a modified Ulcerative Colitis Disease Activity Index (UCDAI) score greater than 1, a UCDAI score greater than 2, a Clinical Activity Index score greater than 4, or a Mayo Clinic score greater than 2 (with a subscore greater than 1). In some embodiments, a human patient is determined to have a UC relapse when the subject experiences rectal bleeding, urgency, and increased stool frequency, e.g., confirmed by endoscopic examination of mucosa. In some embodiments, a human patient is determined to have a UC relapse when there is an increase of 15 or more points for the PUCAI, e.g., between the start of the treatment and 4 weeks after.

To perform the methods of treatment described herein, an effective amount of a 2′-FL compound and composition comprising the same can be administered to a subject in need of the treatment.

An “effective amount” refers to an amount of a 2′-FL compound (e.g., ones as described herein), that alone, or together with further doses, produces the desired response, e.g., elimination or alleviation of symptoms, prevention or reduction the risk of symptom relapse in IBD, a reduction in diarrhea, a reduction of blood in stool, a gain in weight, a reduction of abdominal pain or cramping, an increase in abundance of intestinal microbes that produce short-chain fatty acids (e.g., butyrate), and/or a decrease in intestinal inflammation. The desired response is to inhibit the progression or relapse of the symptoms of the disease. This may involve only slowing the progression of the disease temporarily, although it may involve halting the progression of the disease permanently. In some instances, this may involve only delaying the relapse of the disease temporarily, although it may involve preventing the relapse of the disease permanently. This can be monitored by routine methods. The desired response to treatment of the disease also can be delaying the onset or even preventing the onset of the disease.

Such amounts will depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

For example, an effective amount of a 2′-FL compound (e.g., ones as described herein, either as a free oligosaccharide or in glycoconjugate form as described herein) when administered to a subject in need thereof results in, e.g., by increasing the abundance of intestinal microbes that produce short-chain fatty acids by at least about 10% or more, including, e.g., at least about 20%, at least about 30%, at least about 40%, at least about 50%, at least about 60%, at least about 70%, at least about 80%, at least about 90% or more, as compared to the abundance of short-chain fatty acid-producing intestinal microbes without administration of the 2′-FL compound (either as a free oligosaccharide or in glycoconjugate form as described herein). Examples of intestinal microbes that produce short-chain fatty acids (e.g., butyrate) include, but are not limited to Bifidobacteria, Bacteroides, and/or Parabacteroides. In some embodiments, an effective amount of a 2′-FL compound (e.g., ones as described herein) when administered to a subject in need thereof results in increasing the abundance of intestinal Bifidobacteria by at least about 10% or more, including, e.g., at least about 20%, at least about 30%, at least about 40%, at least about 50%, at least about 60%, at least about 70%, at least about 80%, at least about 90% or more, as compared to the abundance of intestinal Bifidobacteria without administration of the 2′-FL compound (either as a free oligosaccharide or in glycoconjugate form as described herein). Such therapeutic features can be determined by measuring the abundance of fecal microbes (e.g., Bifidobacteria, Bacteroides, and/or Parabacteroides).

In some embodiments, an effective amount of a 2′-FL compound (e.g., ones as described herein, either as a free oligosaccharide or in glycoconjugate form as described herein) when administered to a subject in need thereof results in, e.g., by increasing microbial butyrate production by at least about 10% or more, including, e.g., at least about 20%, at least about 30%, at least about 40%, at least about 50%, at least about 60%, at least about 70%, at least about 80%, at least about 90% or more, as compared to the microbial butyrate production without administration of the 2′-FL compound (either as a free oligosaccharide or in glycoconjugate form as described herein). Such therapeutic feature can be determined by measuring the abundance of fecal short-chain fatty acid including, e.g., butyrate.

In some embodiments, an effective amount of a 2′-FL compound (e.g., ones as described herein, either as a free oligosaccharide or in glycoconjugate form as described herein) when administered to a subject in need thereof results in, e.g., by decreasing intestinal inflammation by at least about 10% or more, including, e.g., at least about 20%, at least about 30%, at least about 40%, at least about 50%, at least about 60%, at least about 70%, at least about 80%, at least about 90% or more, as compared to the intestinal inflammation without administration of the 2′-FL compound (either as a free oligosaccharide or in glycoconjugate form as described herein). Such therapeutic features can be determined by measuring the abundance of, e.g., fecal calprotectin, which is a biomarker of intestinal inflammation.

In some embodiments, an effective amount of a 2′-FL compound (e.g., ones as described herein, either as a free oligosaccharide or in glycoconjugate form as described herein) when administered to a subject in need thereof results in, e.g., by decreasing intestinal inflammation by at least about 10% or more, including, e.g., at least about 20%, at least about 30%, at least about 40%, at least about 50%, at least about 60%, at least about 70%, at least about 80%, at least about 90% or more, as compared to the intestinal inflammation without administration of the 2′-FL compound (either as a free oligosaccharide or in glycoconjugate form as described herein). Such therapeutic features can be determined by measuring the abundance of, e.g., fecal calprotectin, which is a biomarker of intestinal inflammation. Alternatively, such therapeutic features can be determined by measuring the abundance of pro-inflammatory microbes, including, e.g., but not limited to Enterobacteriaceae.

In some embodiments, an effective amount of a 2′-FL compound (e.g., ones as described herein, either as a free oligosaccharide or in glycoconjugate form as described herein) for use in the methods described herein can be equivalent to at least 0.5 mg/day of 2′-fucosyllactose, at least 1 mg/day of 2′-fucosyllactose, at least 2 mg/day of 2′-fucosyllactose, at least 3 mg/day of 2′-fucosyllactose, at least 4 mg/day of 2′-fucosyllactose, at least 5 mg/day of 2′-fucosyllactose, at least 6 mg/day of 2′-fucosyllactose, at least 7 mg/day of 2′-fucosyllactose, at least 8 mg/day of 2′-fucosyllactose, at least 9 mg/day of 2′-fucosyllactose, at least 10 mg/day of 2′-fucosyllactose, at least 11 mg/day of 2′-fucosyllactose, at least 12 mg/day of 2′-fucosyllactose, at least 13 mg/day of 2′-fucosyllactose, at least 14 mg/day of 2′-fucosyllactose, at least 15 mg/day of 2′-fucosyllactose, at least 16 mg/day of 2′-fucosyllactose, at least 17 mg/day of 2′-fucosyllactose, at least 18 mg/day of 2′-fucosyllactose, at least 19 mg/day of 2′-fucosyllactose, or at least 20 mg/day of 2′-fucosyllactose. In some embodiments, an effective amount of a 2′-FL compound (e.g., ones as described herein) for use in the methods described herein can be equivalent to no more than 20 mg/day of 2′-fucosyllactose, no more than 15 mg/day of 2′-fucosyllactose, no more than 10 mg/day of 2′-fucosyllactose, no more than 9 mg/day of 2′-fucosyllactose, no more than 8 mg/day of 2′-fucosyllactose, no more than 7 mg/day of 2′-fucosyllactose, no more than 6 mg/day of 2′-fucosyllactose, no more than 5 mg/day of 2′-fucosyllactose, no more than 4 mg/day of 2′-fucosyllactose, no more than 3 mg/day of 2′-fucosyllactose, or no more than 2 mg/day of 2′-fucosyllactose. Combinations of the above-recited ranges are also included. For example, in some embodiments, an effective amount of a 2′-FL compound (e.g., ones as described herein) for use in the methods described herein can be equivalent to 0.5 mg/day to 20 mg/day of 2′-fucosyllactose, equivalent to 1 mg/day to 20 mg/day of 2′-fucosyllactose, equivalent to 1 mg/day to 15 mg/day of 2′-fucosyllactose, equivalent to 1 mg/day to 10 mg/day of 2′-fucosyllactose, equivalent to 1 mg/day to 8 mg/day of 2′-fucosyllactose, or equivalent to 1 mg/day to 5 mg/day of 2′-fucosyllactose.

In some embodiments where a subject in need of the treatment is at the age of 11-25, an effective amount of a 2′-FL compound (e.g., ones as described herein, either as a free oligosaccharide or in glycoconjugate form as described herein) for use in the methods described herein can be equivalent to 1 mg/day to 20 mg/day of 2′-fucosyllactose, equivalent to 1 mg/day to 15 mg/day of 2′-fucosyllactose, equivalent to 1 mg/day to 10 mg/day of 2′-fucosyllactose, equivalent to 1 mg/day to 8 mg/day of 2′-fucosyllactose, or equivalent to 1 mg/day to 5 mg/day of 2′-fucosyllactose.

In some embodiments, the daily effective amount of a 2′-FL compound (e.g., ones as described herein, either as a free oligosaccharide or in glycoconjugate form as described herein) can be administered in a single daily dose, or divided into multiple doses (e.g., 2-4 doses) for administration at given time intervals during the day. In some embodiments, the daily effective amount of a 2′-FL compound (e.g., ones as described herein) can be administered as a single daily dose in the morning, e.g., alone or in combination with food or beverages. Administration of a 2′-FL compound (e.g., ones as described herein) at any other times during the day is also suitable.

A 2′-FL compound (e.g., ones as described herein, either as a free oligosaccharide or in glycoconjugate form as described herein) can be administered to a subject in need thereof as a single oligosaccharide for treatment of IBD or in combination with at least one additional oligosaccharides (e.g., ones described herein). In some embodiments, a 2′-FL compound (e.g., ones as described herein) is administered to a subject in need thereof as a single oligosaccharide, i.e., the subject is given a 2′-FL compound (e.g., ones as described herein) as the only oligosaccharide, which is not co-used with other oligosaccharides (e.g., ones described herein). In other embodiments, a 2′-FL compound (e.g., ones as described herein) is co-administered with at least one different oligosaccharide. By “co-administered” or “in combination with” is meant that a subject is provided with a 2′-FL compound (e.g., ones as described herein) with a different oligosaccharide during the course of treatment, such as concurrently, consecutively, intermittently, or in other regimens.

A 2′-FL compound (e.g., ones as described herein, either as a free oligosaccharide or in glycoconjugate form as described herein) can be administered as an adjunct to an immune system suppression and/or anti-inflammatory agent, e.g., one being taken by a human IBD patient. An exemplary immune system suppression and/or anti-inflammatory agent includes, but is not limited to an anti-TNF agent. Non-limiting examples of an anti-TNF agent include infliximab, adalimumab, golimumab, natalizumab, vedolizumab, and ustekinumab. In some embodiments, a 2′-FL compound (e.g., ones as described herein) is administered as an adjunct to an anti-TNF agent comprising infliximab and/or adalimumab.

As used herein, the term “adjunct” refers to a first agent being provided as a supplement to a second agent. The first agent can be administered prior to, concurrently with, or after administration of the second agent. In some embodiments, administration of a 2′-FL compound as an adjuvant to an immune system suppression and/or anti-inflammatory agent (e.g., an anti-TNF agent) can provide a synergistic effect on treatment of IBD, including, e.g., alleviating or reducing the risk of relapse in IBD. In some embodiments, administration of a 2′-FL compound as an adjuvant to an immune system suppression and/or anti-inflammatory agent (e.g., an anti-TNF agent) can provide an additive effect on treatment of IBD, including, e.g., alleviating or reducing the risk of relapse in IBD. For example, the therapeutic effect is synergistic when the average duration of remission achieved by the combination of a 2′-FL compound (e.g., ones described herein) and an immune system suppression and/or anti-inflammatory agent (e.g., an anti-TNF agent) is significantly greater than the additive effect ensuing from individual treatment with the same doses of a 2′-FL compound (e.g., ones described herein) and an immune system suppression and/or anti-inflammatory agent (e.g., an anti-TNF agent). In some embodiments, the synergistic therapeutic effect increases the average duration of remission by at least 20%, 30%, 40%, 50%, 60%, 70%, 80%, 90% or more.

When a 2′-FL compound (e.g., ones described herein) is co-used with a second agent (e.g., other oligosaccharides as described herein or an immune system suppression and/or anti-inflammatory agent (e.g., an anti-TNF agent)), it may be formulated together with the second agent in a single composition, which may be in any suitable form as described herein (e.g., powder or tablets for oral administration). Alternatively, the 2′-FL compound (e.g., ones described herein) and the second agent (e.g., other oligosaccharides as described herein or an immune system suppression and/or anti-inflammatory agent (e.g., an anti-TNF agent)) may be formulated separately.

Administration of IBD treatment described herein may be accomplished by any method known in the art (see, e.g., Harrison's Principle of Internal Medicine, McGraw Hill Inc., 18^{th} ed., 2011). For combined treatment, each agent can be administered via the same route or different routes. Administration may be local or systemic. Administration may be, for example, parenteral (e.g., intravenous, intraperitoneal, subcutaneous, intra-arterial or intradermal), or oral. Compositions for different routes of administration are well known in the art (see, e.g., Remington: The Science and Practice of Pharmacy, Pharmaceutical Press, 22^{nd} ed., 2012). The compositions may also be formulated as modified release dosage forms, including delayed-, extended-, prolonged-, sustained-, pulsed-, controlled-, accelerated- and fast-, targeted-, programmed-release, and gastric retention dosage forms. These dosage forms can be prepared according to conventional methods and techniques known to those skilled in the art. Dosage will depend the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. Dosage can be determined by the skilled artisan.

In some embodiments, a 2′-FL compound (e.g., ones described herein) and/or a second agent (e.g., other oligosaccharide(s) as described herein or an immune system suppression and/or anti-inflammatory agent (e.g., an anti-TNF agent) can be administered orally. Oral administration also includes buccal, lingual, and sublingual administration. In some embodiments, compositions comprising a 2′-FL compound (e.g., ones described herein) may be provided in solid, semisolid, or liquid composition (e.g., pharmaceutical composition or dietary supplement) for oral administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the compositions may contain one or more pharmaceutically acceptable or edible carriers or excipients, including, but not limited to, binders, fillers, diluents, disintegrants, wetting agents, lubricants, glidants, coloring agents, dye-migration inhibitors, sweetening agents, and flavoring agents.

In some embodiments, a 2′-FL compound (e.g., ones described herein) can be administered by injection (e.g., parenterally such as intravenously or intraperitoneally). Preparations for parenteral administration include sterile aqueous or non-aqueous solutions, suspensions, and emulsions. Examples of non-aqueous solvents or vehicles are propylene glycol, polyethylene glycol, vegetable oils, such as olive oil and corn oil, gelatin, and injectable organic esters such as ethyl oleate. Such dosage forms may also contain one or more of a preserving agent, a wetting agent, an emulsifying agent and a dispersing agent. The dosage forms may be sterilized by, for example, filtration of the composition, by irradiating the composition, or by heating the composition. They can also be manufactured using sterile water, or some other sterile injectable medium, prior to use.

In some embodiments, the method further comprises taking actions other than or in addition to an IBD treatment described herein. In some embodiments, the method further comprises monitoring development of an IBD symptom of a subject who is at risk for IBD, or monitoring the effectiveness of the treatment. The monitoring may comprise a physical examination, endoscopy, and/or stool sample examination, e.g., for assessing intestinal inflammation and/or intestinal microbiota. If the subject is not responsive to an administered dose of a 2′-FL compound (e.g., ones described herein, either as a free oligosaccharide or in glycoconjugate form as described herein), a physician can increase the dose of the 2′-FL compound, e.g., based on the medical and/or physical condition of the subject, provided that the increased dose does not cause significant gastrointestinal symptoms such as bloating, abdominal pain, nausea, loose stools, and/or gassiness.

### IV. Kits for Use in IBD Treatment

Another aspect of the present disclosure relates to kits for use in IBD treatment described herein. Accordingly, in some embodiments, such a kit can comprise a 2′-FL compound (e.g., ones described herein, either as a free oligosaccharide or in glycoconjugate form as described herein), or a pharmaceutical composition comprising the same, or a dietary supplement comprising the same.

In some embodiments, the kit can comprise instructions for use in accordance with any of the methods described herein. The instructions can comprise a description of administration of a 2′-FL compound (e.g., ones described herein, either as a free oligosaccharide or in glycoconjugate form as described herein), or a pharmaceutical or a dietary supplement composition comprising the same, for IBD treatment. The instructions relating to a 2′-FL compound (e.g., ones described herein), or a pharmaceutical or dietary supplement composition comprising the same, generally include information as to dosage, dosing schedule, and route of administration for the intended treatment. Such instructions may also include recommended weight-based dosages and/or age-based dosages.

Instructions supplied in the kits described herein are typically written instructions on a label or package insert (e.g., a paper sheet included in the kit), but machine-readable instructions (e.g., instructions carried on a magnetic or optical storage disk) are also acceptable. The label or package insert indicates that the composition is used for IBD treatment in subjects. In some embodiments, the label or package insert may indicate that the composition is suitable for use in specific groups of subjects, e.g., as described herein. For example, the label or package insert may indicate that the composition is suitable for use in human IBD patients (e.g., human CD or UC patients) who has undergone or is on an immune system suppression and/or anti-inflammatory therapy. In some embodiments, the label or package insert may indicate that the composition is suitable for use in human IBD patients (e.g., human CD or UC patients) who are receiving stable maintenance anti-TNF therapy. Instructions may be provided for practicing any of the methods described herein.

A 2′-FL compound (e.g., ones described herein), or a pharmaceutical or dietary supplement composition comprising the same in the kit may be in suitable packaging. Suitable packaging includes, but is not limited to, vials, bottles, jars, flexible packaging (e.g., sealed Mylar or plastic bags or paper bags with a polyethylene liner), and the like. The packaging may be in unit doses, bulk packages (e.g., multi-dose packages) or sub-unit doses.

Kits may optionally provide additional components such as buffers and interpretive information. Normally, the kit comprises a container and a label or package insert(s) on or associated with the container.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Dosing and Efficacy of 2′-Fucosyllactose in Inflammatory Bowel Disease

The Inflammatory Bowel Diseases (IBD), Crohn Disease (CD) and Ulcerative Colitis (UC), are chronic and debilitating disorders with peak incidence in the second and third decades of life (Kaplan et al., Gastroenterology, 152:313-321 e2, 2017). While considerable progress has been made in optimizing medications to achieve remission, relapse is common and unpredictable (Minar et al., Inflamm Bowel Dis, 22:2641-2647, 2016; Minar et al., J Pediatr Gastroenterol Nutr, 62:715-22, 2016). Altered microbiota likely drive gut inflammation and clinical relapses (De Cruz et al., J Gastroenterol Hepatol, 30:268-78, 2015; Gevers et al., Cell Host Microbe, 15:382-92, 2014; Rajca et al., Inflamm Bowel Dis, 20:978-86, 2014; Varela et al., Aliment Pharmacol Ther, 38:151-61, 2013; Wills et al., PloS One, 9:e90981, 2014). Suppression of mucosal inflammation with infliximab (monoclonal anti-TNF antibody) only partially corrects this dysbiosis (Lewis et al., Cell Host Microbe, 18:489-500, 2015).

A critical barrier to progress in the IBD field has been the lack of evidence-based approaches to directly modulate the microbiota to prevent clinical relapse (Kaplan et al., Gastroenterology, 152:313-321 e2, 2017; Sartor et al., Gastroenterology, 152:327-339 e4, 2017). While a wide variety of prebiotics are commercially available, the lack of controlled dose-finding studies with appropriate clinical and microbial endpoints precludes informed decision-making regarding their use (Ghouri et al., Clin Exp Gastroenterol, 7:473-87, 2014).

While TNF-alpha inhibitors are effective, the therapy has high potential toxicity and does not directly address the dysbiosis (microbial dysregulation) that is a hallmark of IBD (Lewis et al., Cell Host Microbe, 18:489-500, 2015). Presented herein relates to use of the prebiotic human milk oligosaccharide, 2′-fucosyllactose (2′-FL) for maintaining remission in IBD patients. A pilot dose-finding study is used to assess if 2′-FL supplementation in IBD patients is safe and well tolerated, while increasing abundance of short-chain fatty acids (SCFA) producing microbiota and reducing gut inflammation. The study utilizes state of the art genomic approaches to assess the response. This study provides critical safety and efficacy data. The studies support a fundamental shift in clinical practice towards personalized microbial therapeutic interventions to maintain clinical remission in the growing worldwide population of IBD patients.

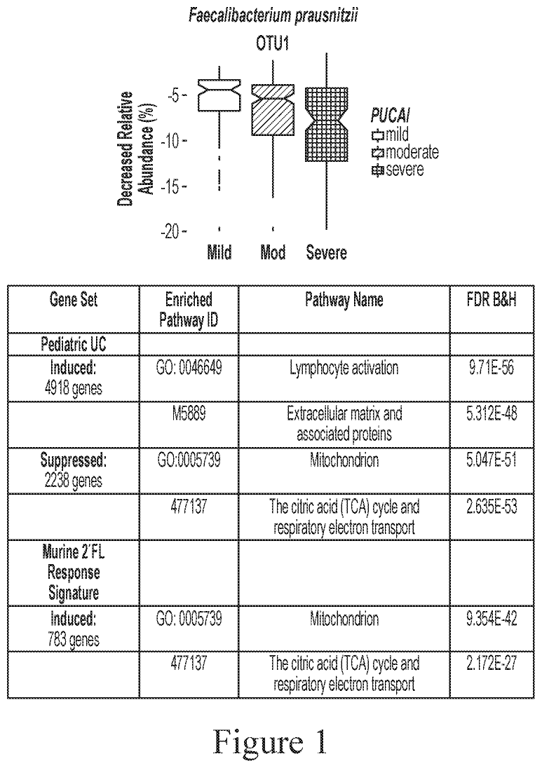

The multi-center RISK pediatric CD and PROTECT pediatric UC inception cohort studies have been conducted to test for genomic and microbial factors associated with clinical outcomes (Gevers et al., Cell Host Microbe, 15:382-92, 2014; Kugathasan et al. Lancet, 389:1710-1718, 2017; Haberman et al., J Clin Invest, 124:3617-33, 2014). It was found that early anti-TNF therapy reduced progression to internal penetrating, but not stricturing, complications in the RISK CD cohort (Kugathasan et al. Lancet, 389:1710-1718, 2017). An imbalance between low expression of ileal genes regulating mitochondrial function, and high expression of ileal genes driving extra-cellular matrix (ECM) production, was found in patients who progressed to strictures (Kugathasan et al. Lancet, 389:1710-1718, 2017). Distinct microbial taxa were in turn associated with complications (Kugathasan et al. Lancet, 389:1710-1718, 2017). Within the PROTECT UC cohort, taxa associated with disease severity were predominantly from the Ruminococcaceae and Lachnospiraceae family, including two common commensals: Faecalibacterium prausnitzii, a known short chain fatty acid (SCFA) producer and Dorea formicigenerans, which is a member of the Clostridium cluster XIV (FIG. 1 and data not shown). The rectal global pattern of gene expression included induction of lymphocyte activation and associated extra-cellular matrix (ECM) responses, while a gene program regulating mitochondrial function was profoundly suppressed (FIG. 1). Remission rates with corticosteroids (CS) were inversely correlated with the degree of mitochondrial pathway dysregulation, from 32% in the highest quartile for dysregulation to 71% in the lowest quartile (p=0.0004). 2′-FL supplementation enhanced microbial alpha diversity, including a specific increase in SCFA-producing Parabacteroides (Mezoff et al., Am J Physiol Gastrointest Liver Physiol, 310:G427-38, 2016). The predominant small bowel gene signature induced by 2′-FL was one for enhanced mitochondrial function (FIG. 1) (Mezoff et al., Am J Physiol Gastrointest Liver Physiol, 310:G427-38, 2016). In this study, it is sought to assess if 2′-FL supplementation in IBD patients can enhance intestinal mitochondrial function via induction of SCFA-producing microbes as a novel therapeutic target.

### Define Dose Dependent Safety, Tolerability, and Efficacy of 2′-FL as a Dietary Supplement in IBD.

1, 5, or 10 gm 2′-FL is provided as a daily dietary supplement to pediatric and young adult IBD patients in stable remission receiving infliximab or adalimumab anti-TNF therapy. Safety and tolerability are assessed using validated clinical disease activity indices, electronic symptom trackers, fecal metabolite assays, and fecal calprotectin. Efficacy is assessed by determining the dose dependent effect of 2′-FL upon increased fecal Bifidobacteria and decreased fecal calprotectin abundance, as a biomarker of mucosal inflammation.

### Determine the Effect of 2′-FL Supplementation Upon the Gut Microbial Community and Associated SCFA Production.

Established genomic and metabolomics approaches are utilized to test the effect of a range of 2′-FL doses compared to glucose placebo upon the gut microbial community and associated metabolic functions with a focus upon SCFA production.

### Study Design

A single center randomized dose-ranging study of 2′-FL as a dietary supplement in pediatric and young adult IBD patients receiving stable maintenance infliximab or adalimumab (anti-TNF) therapy is conducted. The primary objective of this study is to obtain 2′-FL dose-dependent safety and efficacy data to guide design of a larger multi-center placebo-controlled RCT. Inclusion criteria includes male and female CD and UC patients aged 11 and above currently in corticosteroid-free remission receiving stable anti-TNF maintenance therapy.

Patients are excluded if they have experienced a clinical relapse during the previous six months, or received antibiotics, probiotics, or prebiotics during the previous month. Three-day diet diaries are used to determine whether differences in usual diet are associated with differential responses, and participants are encouraged to maintain a stable diet. The study timeline and procedures are summarized in Table 1.

TABLE 1
Study Timelines and Procedures

| | Baseline | Week 4 | Week 8 | Week 20 |
|---|---|---|---|---|
| Study visits | X | X | X | X |
| Study coordinator call | X | X | X | X |
| 2′-FL supplementation | | X | X | |
| Three day diet dairy | | X | X | |
| wPCDAI or PUCAI | X | X | X | X |
| Orchestra symptom tracker | X | X | X | X |
| Safety labs | | X | X | |
| Plasma Cytokines | X | X | X | X |
| Fecal calprotectin | X | X | X | X |
| Fecal 16S microbial | X | X | X | X |
| community profiles | | | | |

Coordinator calls occur prior to each of the four study visits, and at weeks 5, 6, and 7. Data from the Orchestra symptom tracker are obtained weekly with the exception of between weeks 4 and 8 when it is obtained daily. Safety labs include CBC, CMP, PT/INR, and U/A.

Patients complete a 4 week run-in period to collect baseline data for gastrointestinal (GI) symptoms, plasma cytokines, fecal calprotectin, and the fecal microbial community. A smartphone-based symptom tracker is utilized to track patient-reported measures of 2′-FL tolerability including abdominal pain, nausea, loose stools, and gassiness. These parameters were modestly increased in healthy adults who received the 20 g dose of 2′-FL in a recent randomized controlled trial (RCT), but did not vary at lower doses of 2′-FL (Elison et al., Br J Nutr, 116:1356-1368, 2016). The severity of each of these four symptoms are reported on a ten-point Likert scale ranging from (1) no symptoms to (10) severe symptoms, and an average score is computed for each participant for the baseline to week 4, week 4 to week 8, and week 8 to week 20 time periods. Participants are randomized to consume one of three daily doses of 2′-FL for a period of 4 weeks. The recent dose finding RCT of 2′-FL in healthy adults found that it was safe and well tolerated at doses of 5, 10, and 20 g/d for two weeks, with no changes in fecal calprotectin as a biomarker of intestinal inflammation (Elison et al., Br J Nutr, 116:1356-1368, 2016). A three-fold increase in Bifidobacterium was observed with the 10 g dose (Elison et al., Br J Nutr, 116:1356-1368, 2016). Therefore, 1, 5, and 10 g/d of 2′-FL are tested over 4 weeks in 5 CD and 5 UC subjects per dosing group. 2′-FL powder are provided in single dose packets. Participants are asked to dissolve the powder in water immediately prior to consumption with breakfast each morning. Patient self-report data is obtained for 2′-FL intake between weeks 4 and 8 when they are asked to record daily symptoms and 2′-FL consumption. Patients and their parents are also provided with free-of-charge automatically generated cellular telephone text and/or email reminder prompts in an effort to improve 2′-FL adherence to acceptable levels, which are defined as consumption of the randomized dose on at least 24 out of 30 treatment days. They then complete a 12 week follow-up period to determine the stability of any changes detected for clinical disease activity, self-reported GI symptoms, plasma cytokines, and fecal calprotectin or microbiota during the four week period of supplementation.

### Study End Points

The primary safety endpoint is clinical relapse using a validated measure of disease activity, the weighted Pediatric Crohn Disease Activity Index (wPCDAI) for CD patients and the Pediatric Ulcerative Colitis Activity Index (PUCAI) for UC patients (Turner et al., Inflamm Bower Dis, 15:1218-23, 2009; Turner et al., J Pediatr Gastroenterol Nutr, 64:254-260, 2017). Clinical relapse is defined as an increase of more than 20 points for the wPCDAI, and 15 points for the PUCAI, between weeks 4 and 8 (Turner et al., Inflamm Bower Dis, 15:1218-23, 2009; Turner et al., J Pediatr Gastroenterol Nutr, 64:254-260, 2017). If more than two subjects in a dosing group experience clinical relapse, or an overall increase in the GI symptoms tolerability score is observed, it is concluded that that dose was not safe and well tolerated. The secondary safety endpoints are the GI symptom score for tolerability collected using a symptom tracker, and fecal calprotectin. The primary efficacy endpoint is the increase in fecal Bifidobacterium genus abundance with 2′-FL supplementation within each dosing group between weeks 4 and 8. The Illumina MiSeq platform is used to generate a 16S-DNA profile at an average depth of 20,000 paired-end filtered reads per sample and the primer set targeting the V4 (515F/806R) region is used (Gevers et al., Cell Host Microbe, 15:382-92, 2014). Read processing and error correction are performed on the high-performance computing cluster using the DADA2 package and algorithm in R shown to be more sensitive and specific than percent similarity (i.e. OTU) clustering methods (Callahan et al., Nat Methods, 13:581-3, 2016). The secondary efficacy endpoint is the reduction in fecal calprotectin as a biomarker of intestinal inflammation between week 4 and 8.

### Statistical Analysis

The primary analysis is on a per protocol basis, including only patients who consumed at least 24 out of 30 2′-FL doses to which they were randomized (Elison et al., Br J Nutr, 116:1356-1368, 2016). Differences in Bifidobacterium abundance, fecal calprotectin, GI symptom tolerability score, and plasma cytokines before and after supplementation are tested using mixed ANOVA (or the non-parametric equivalent) with Bonferroni's multiple comparisons correction. Models are fit via mixed-effects regression with within-subject contrasts comparing the change in response between weeks 4 to 8 of primary interest. Differences in week 8 clinical relapse rates between each of the 2′-FL intervention groups are compared using Fisher's exact test. Within-dose comparisons for the number of relapses between weeks 4 and 8 are conducted using exact test for paired data. Based upon the recent RCT in healthy adults (Elison et al., Br J Nutr, 116:1356-1368, 2016), it is expected to detect a two-fold increase in fecal Bifidobacterium abundance at week 8 following 2′-FL supplementation at the 10 g dose, and a two-fold decrease in fecal calprotectin. Ordinations and statistical learning approaches for high-dimensional data are used to identify differences in microbial community structure in response to 2′-FL supplementation.

### Consideration of Relevant Biologic Variables

Biologic variables which may influence 2′-FL safety and efficacy include age, sex, race/ethnicity, FUT2 secretor status, IBD diagnosis of CD or UC, mucosal inflammation as measured by fecal calprotectin, and the baseline microbial community (Lewis et al., Cell Host Microbe, 18:489-500, 2015; Currier et al., Clin Infect Dis, 60:1631-8, 2015; Payne et al., JAMA Pediatr, 169:1040-5, 2015; Tong et al., ISME J, 8:2193-206, 2014; Wacklin et al., PLoS One, 6:e20113, 2011). Equal numbers of males and females ages 11 and above, and Caucasian (90%) and African-American (10%) subjects in proportion to the overall CCHMC IBD population are enrolled. Younger children are excluded pending identification of any unanticipated safety signals. Effects of age, sex, race, CD vs UC diagnosis, week 4 fecal calprotectin and microbiota, and FUT2 secretor status are tested in an exploratory manner to guide design of the multi-center RCT.

### Sample Size

The sample size of 10 participants per 2′-FL dosing group is based on the recent dose-finding RCT in healthy adults, in which mean(SD) fecal Bifidobacterium relative abundance increased from 7(2) at baseline to 20(4) after two weeks at the 10 g dose (Elison et al., Br J Nutr, 116:1356-1368, 2016). Based upon the recent reports, it is expected to observe greater variability in fecal Bifidobacterium abundance in CD patients (Gevers et al., Cell Host Microbe, 15:382-92, 2014; Kugathasan et al. Lancet, 389:1710-1718, 2017). Thus, with 10 participants per 2′-FL dosing group, there is 80% power at α=0.0167 to detect a mean increase of 5(4) in fecal Bifidobacterium abundance within each dosing group accounting for multiple testing. Should greater variability in Bifidobacterium abundance be observed, 80% power is retained to detect a mean increase of 10 in the relative abundance if the SD of the mean difference is twice as high as anticipated. A 10% drop-out rate is assumed, and so 33 participants are enrolled.

### Recruitment, Follow-Up, and Retention

Subjects are enrolled from the IBD population age 11 and above currently in sustained remission receiving infliximab or adalimumab maintenance therapy. Patient visits are mandated at baseline and weeks 4, 8, and 20. The study coordinator contacts the patients by phone prior to each study visit, and at weeks 5, 6, and 7 to support retention and adherence to the study procedures. If an emerging signal for lack of tolerability is detected for a 2′-FL dose early stopping of randomization to that dose is implemented.

### Example 2: Pilot and Feasibility Study of 2′-FL as a Dietary Supplement in Pediatric and Young Adult IBD Patients Receiving Stable Maintenance Anti-TNF Therapy

Prebiotics studied in prior IBD RCTs have included oligofructose-enriched inulin (OF-IN), fructo-oligosaccharides (FOS), galacto-oligosaccharides (GOS), and psyllium (Benjamin et al., Gut, 60:923-9, 2011). A 4 week RCT of 15 g FOS in 103 active CD patients did not show a benefit compared to placebo for clinical response (Benjamin et al., Gut, 60:923-9, 2011).

### Microbial SCFA Production and Epithelial Mitochondrial Function

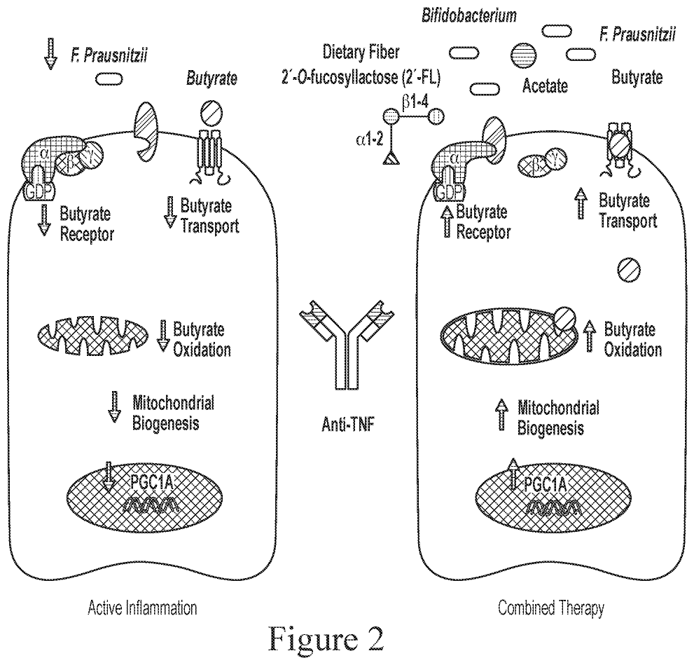

The microbial SCFA metabolite butyrate regulates intestinal epithelial cell (IEC) function via two mechanisms, as an energy source for oxidative phosphorylation and ATP production, and as a regulator of gene transcription via histone deacetylase (HDAC) activity (Donohoe et al., Cell Metab, 13:517-26, 2011; Kaiko et al., Cell, 167:1137, 2016). Colonocytes isolated from germ-free mice exhibit reduced oxidative phosphorylation and ATP production (Donohoe et al., Cell Metab, 13:517-26, 2011). Consequences include diarrhea and poor weight gain. The defect in colonocyte mitochondrial function can be rescued by mono-association with a butyrate-producing bacterial strain, Butyrivibrio fibrisolve (Donohoe et al., Cell Metab, 13:517-26, 2011). The transcriptional co-activator, Peroxisome Proliferator-activated Receptor-γ Coactivator 1-α (PGC1A) is the central regulator of mitochondrial biogenesis in intestinal epithelia (Cunningham et al., J Biol Chem, 291: 10184-200, 2016). Targeted IEC PGC1A deletion causes barrier dysfunction and increased severity of colitis following dextran sodium sulfate administration (Cunningham et al., J Biol Chem, 291: 10184-200, 2016). Inflammatory suppression of EC genes regulating butyrate transport, signaling, and mitochondrial oxidation is largely corrected by infliximab anti-TNF therapy in IBD patients who experience mucosal healing (De Preter et al., Inflamm Bowel Dis, 4:e30, 2012). However, suppression of mucosal inflammation by infliximab was not associated with increased Bifidobacterium abundance in a recent pediatric CD study (Lewis et al., Cell Host Microbe, 18:489-500, 2015). The studies discussed herein determine whether 2′-FL administration is safe and well tolerated in CD and UC patients in stable remission on anti-TNF therapy, and exerts a dose-dependent effect upon butyrate-producing microbiota as illustrated in FIG. 2.

While several IBD therapies targeting inflammatory cytokines have reached the clinic, no mechanistically based approach to directly modulate the microbiota is in use (Sartor et al., Gastroenterology, 152:327-339 e4, 2017). This study challenges the current clinical practice paradigm by testing for the first time adjunct microbial therapy in the context of anti-TNF immune suppression. Basic and translational research has firmly established the role of the microbiota and associated metabolites in the pathogenesis of IBD (Sartor et al., Gastroenterology, 152:327-339 e4, 2017). It is now understood that environmental factors which have contributed to the rising incidence of the disease across the globe have largely done so by triggering pro-inflammatory microbial shifts which interact with host genetic variation to drive chronic mucosal inflammation¹. In this study, 2′-FL, a prebiotic trisaccharide synthesized by the FUT2 gene enzyme, is used as a therapeutic agent in IBD. Because a polymorphism in the FUT2 gene (inactivating mutation, or non-secretor status) is associated with increased risk of CD, the optimal dose of 2′-FL in relation to FUT2 secretor status is also determined to inform personalized clinical trials which can account for the source of patient variability in response (McGovern et al., Hum Mol Genet, 19:3468-76, 2010). State of the art electronic symptom trackers, microbial high-throughput sequencing methods, and fecal metabolite assays are utilized to precisely assess the response (Integrative HMPRNC, Cell Host Microbe, 16:276-89, 2014). Results of the studies discussed herein support a fundamental shift in clinical practice towards personalized microbial therapeutic interventions.

### Preliminary Studies

Across the 95 sites participating in the lmproveCareNow (ICN) pediatric IBD Quality Improvement (QI) network, 47% of patients relapsed in the past year (improvecarenow.org). At the Cincinnati site, 37% of patients receiving infliximab or adalimumab anti-TNF therapy relapsed despite optimal therapeutic drug monitoring and dosing (Minar et al., Inflamm Bowel Dis; 2016; Minar et al., J Pediatr Gastroenterol Nutr, 62:715-22, 2016). In addition to adverse effects upon quality of life and school and work productivity, relapses increase cost of care. Based upon a recent cost effectiveness analysis, annual pharmacy charges for adalimumab or infliximab therapy in the United States range from $27,664 to $92,300 per year (Yokomizo et al., BMJ Open Gastroenterol, 3:e000093, 2016). At the Cincinnati site, median annual cost of care increased by $16,862 amongst patients on anti-TNF therapy who relapsed. By comparison, it is estimated that the annual cost for 2′-FL at the 10 g daily dose is about $500, providing a highly cost-effective adjunct therapeutic option.

2′-FL promotes the growth of Bifidobacterium, which via acetate cross-feeding of F. prausnitzii and other beneficial microbes enhances butyrate production (Rios-Covian et al., FEMS Microbial Lett, 362, 2015; Yu Z T et al., Glycobiology, 23:1281-92, 2013). Conversely, 2′-FL does not support the growth of Enterobacter spp. or Escherichia, which are increased in IBD patients with more severe symptoms (Gevers et al., Cell Host Microbe, 15:382-92, 2014; Morgan et al., Genome Biol, 13:R79, 2012; Yu Z T et al., Glycobiology, 23:1281-92, 2013). In addition to these prebiotic effects, 2′-FL also exerts direct anti-inflammatory effects in the gut by inhibiting pathogen adhesion, and suppressing epithelial inflammatory responses to bacterial products (He et al., Gut, 65:33-46, 2016; Yu et al., J Nutr, 146:1980-1990, 2016). It was reported that 2′-FL promotes weight gain in mice following ileocecal resection (Mezoff et al., Am J Physiol Gastrointest Liver Physiol, 310:G427-38, 2016). This was associated with expansion of Parabacteroides and induction of an intestinal gene signature for mitochondrial function (Mezoff et al., Am J Physiol Gastrointest Liver Physiol, 310:G427-38, 2016). Pre-clinical safety studies of 2′-FL in rats have established a No Observed Adverse Effect Level (NOAEL) of 5 gm/kgbw/day for both males and females (Goulet et al., Regul Toxicol Pharmacol, 68:59-69, 2014). Infants fed a 2′-FL supplemented formula exhibited improved growth velocity and lower plasma cytokine profiles compared to infants fed a control formula (Goehring et al., J Nutr, 146:2559-2566, 2016; Marriage et al., J Pediatr Gastroenterol Nutr; 61:649-58, 2015). A recent RCT in healthy adults reported that 2′-FL was safe and well tolerated across a range of doses from 5 gm to 20 gm per day, and promoted expansion of Bifidobacterium and reduction in Proteobacteria (Elison et al., Br J Nutr, 116:1356-1368, 2016). However, whether 2′-FL exerts similar benefits in an IBD population is not known. Prior studies have demonstrated profound shifts in 2′-FL target microbiota, and reduction in colonic butyrate absorption and oxidative metabolism, in the setting of active mucosal inflammation (Gevers et al., Cell Host Microbe, 15:382-92, 2014; Morgan et al., Genome Biol, 13:R79, 2012; De Preter et al., Inflamm Bowel Dis, 18:1127-36, 2012). These alterations in the microbial niche and host butyrate metabolism in the setting of active mucosal inflammation could reduce 2′-FL treatment benefits. This may account for mixed results of topical butyrate enema therapy in the setting of active colitis (Scheppach et al. Gastroenterology, 103:51-6, 1992). Therefore, a more effective approach is to first suppress mucosal inflammation with anti-TNF therapy, and then utilize 2′-FL supplementation to promote expansion of SCFA-producing microbiota to enhance maintenance of remission (FIG. 2).

Microbial Shifts Associated with Disease Complications and Treatment Responses in CD

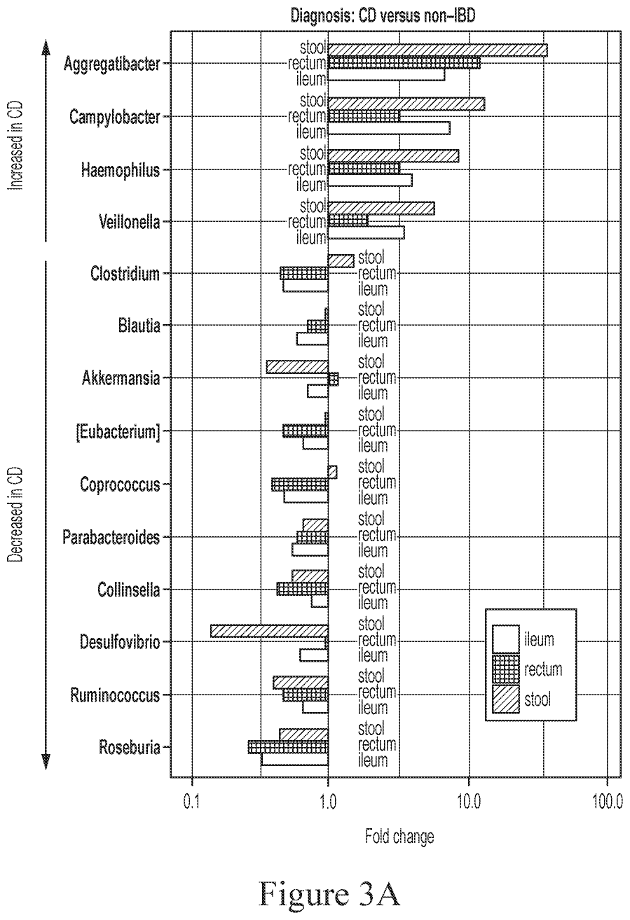

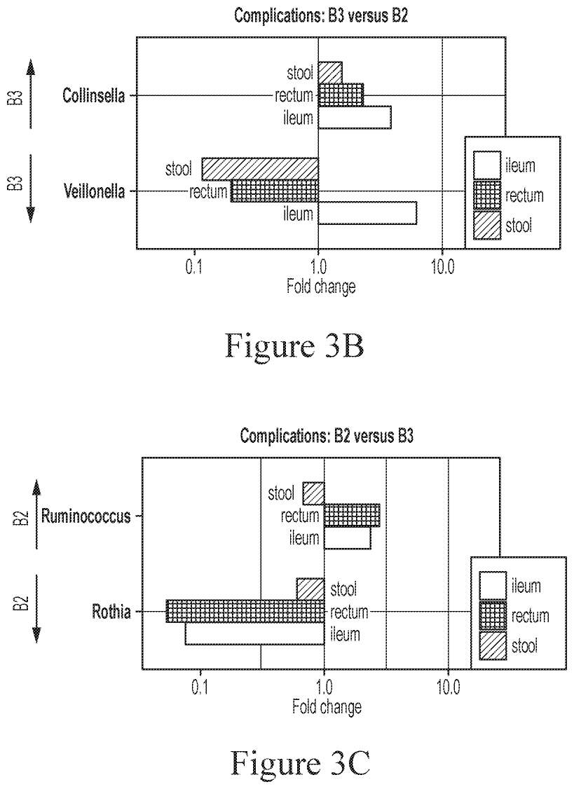

The multi-center CCFA sponsored RISK inception cohort study was conducted to test for clinical, demographic, genomic, microbial, and immune factors associated with initial treatment responses and subsequent development of disease complications during 36 months follow-up in 913 pediatric CD patients enrolled at diagnosis, prior to therapy (Gevers et al., Cell Host Microbe, 15:382-92, 2014; (Kugathasan et al. Lancet, 389:1710-1718, 2017; (Haberman et al., J Clin Invest, 124:3617-33, 2014). The ileal and rectal global pattern of gene expression, and ileal, rectal, and fecal microbial community were determined using high-throughput sequencing in a representative subset of 243 and 22 CD patients, respectively (Gevers et al., Cell Host Microbe, 15:382-92, 2014; (Kugathasan et al. Lancet, 389:1710-1718, 2017; (Haberman et al., J Clin Invest, 124:3617-33, 2014). Expansion of pro-inflammatory genera was identified including Veillonellaceae in conjunction with contraction of butyrate producing Blautia, Parabacteroides, and Roseburia in the treatment naive ileum and rectum (FIG. 3A) (Gevers et al., Cell Host Microbe, 15:382-92, 2014; (Kugathasan et al. Lancet, 389:1710-1718, 2017; (Haberman et al., J Clin Invest, 124:3617-33, 2014). Distinct taxa were in turn associated with progression to B2 stricturing or B3 internal penetrating complications, suggesting a role in modulating host biology (FIGS. 3B and 3C). A multivariate logistic regression model which included baseline microbial abundance was superior to one including only clinical, demographic, and genomic factors in predicting steroid and surgery free remission (SFR) six months after diagnosis (Haberman et al., J Clin Invest, 124:3617-33, 2014). In this model, the relative abundance of Blautia and Veillonellaceae was associated with the likelihood of achieving SFR after accounting for anti-TNF exposure (Haberman et al., J Clin Invest, 124:3617-33, 2014). Importantly, data supported a model in which these pro- and anti-inflammatory microbes co-excluded each other, with concurrent antibiotic use exacerbating the dysbiosis (Gevers et al., Cell Host Microbe, 15:382-92, 2014).

Microbial Shifts Associated with Disease Severity in UC

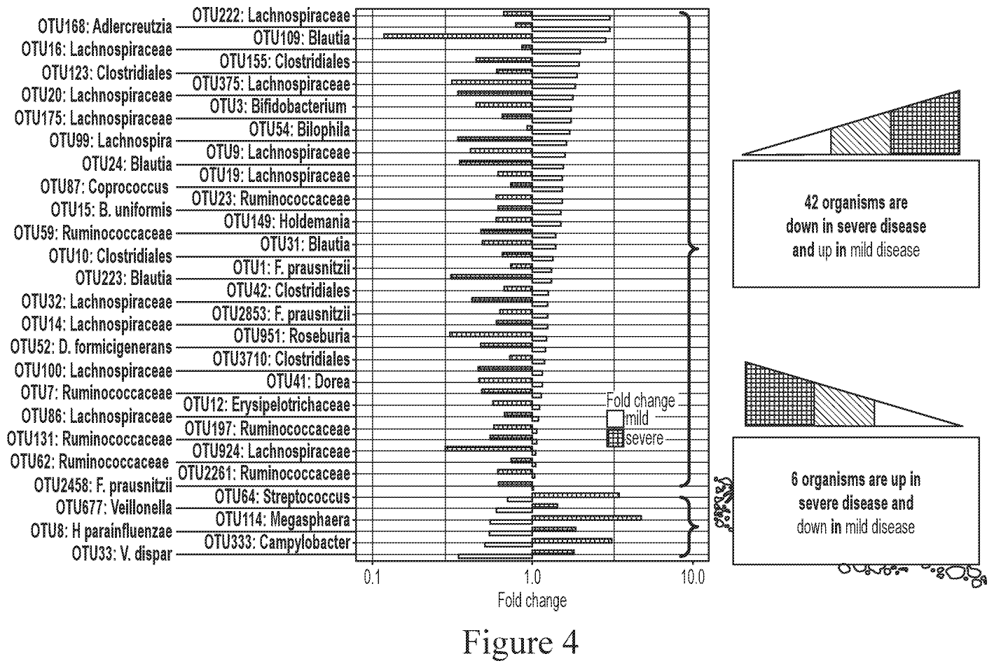

Following up on the success of the RISK study, the NIH/NIDDK sponsored PROTECT inception cohort study is conducted to test for clinical, demographic, genomics, microbial, and immune factors associated with the achieving SFR with mesalamine alone in 431 pediatric UC patients enrolled at diagnosis, prior to therapy. The rectal global pattern of gene expression, and rectal and fecal microbial community, have been determined using high-throughput sequencing in a representative subset of 206 and 371 UC patients, respectively. In total, 48 operational taxonomic units (OTUs) were associated with disease severity and exhibited a continuous increase or decrease with increasing disease severity (FDR threshold: 0.5) (FIG. 4). Most OTUs were negatively correlated with disease severity, indicating that a loss of these bacterial taxa is associated with exacerbation of UC. Predominantly, these OTUs are from the Ruminococcaceae and Lachnospiraceae family, including two common commensals: F. prausnitzii, a known SCFA producer and Dorea formicigenerans, which is a member of the Clostridium cluster XIV. An increase in six OTUs was associated with increased severity representing many Veillonellaceae organisms, such as Veillonella dispar and Megasphaera. These data from the large prospective inception cohort studies provide support for testing the dose dependent effects of 2′-FL in modulating SCFA-producing microbiota in IBD.

An Ileal Gene Signature for Mitochondrial Dysfunction is Associated with Disease Complications in Pediatric CD

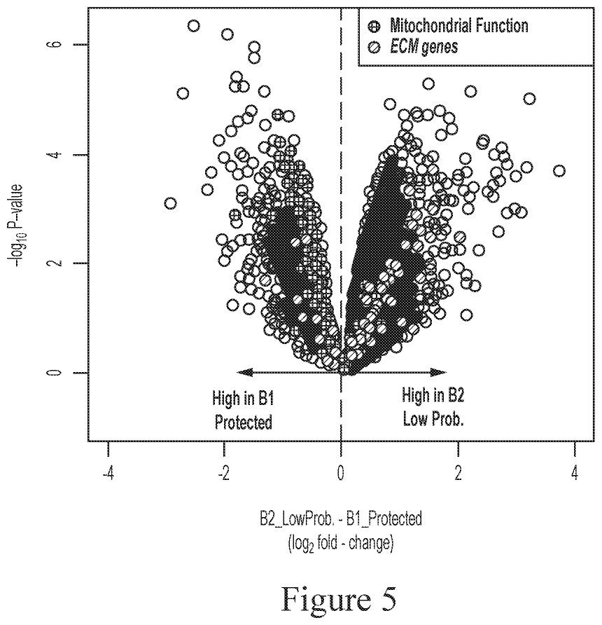

While there was considerable heterogeneity in the ileal global pattern of gene expression within the RISK cohort, comparisons between groups revealed significant differences in gene expression (Kugathasan et al. Lancet, 389:1710-1718, 2017; Haberman et al., J Clin Invest, 124:3617-33, 2014). Analyses identified enrichment for a mitochondrial function gene signature in patients otherwise at high risk for a B2 stricturing complication remaining complication-free (B1 protected, FIG. 5). Conversely, enhancement of an extra-cellular matrix (ECM) gene signature was detected in predicted low risk patients who nevertheless progressed to a stricture (B2 Low Probability). Genes involved in the mitochondrial respiratory chain (GO pathway:0022900 and GO pathway:0045333; in dark shade) were upregulated in “B1 Protected”, whereas genes involved in ECM production (GO pathway:0005201; in light shade) were upregulated in “B2 Low Probability”. A multivariate logistic regression model for disease complications which included these gene signatures was superior to one including only clinical, demographic, and serologic factors (Kugathasan et al. Lancet, 389:1710-1718, 2017). Higher expression of the ileal mitochondrial function gene signature was associated with a lower likelihood of developed a stricturing complication in the model (HR(95^{th}Cl):0.69(0.51,0.94), p=0.019), while higher expression of the ECM gene signature was associated with an increased likelihood of developed a stricturing complication (HR(95^{th}Cl):1.7(1.12,2.57), p=0.012). These data indicate that approaches to boost intestinal epithelial cell (IEC) mitochondrial function, such as expansion of butyrate-producing microbiota by 2′-FL, can improve CD treatment responses and outcomes.

A Rectal Gene Signature for Mitochondrial Dysfunction Associated with Clinical Severity and Treatment Response in Pediatric UC is Regulated by 2′-FL Supplementation in Mice

A similar analysis of rectal gene expression was conducted for patients enrolled in the PROTECT UC cohort study. Genes differentially expressed between UC patients and controls at ≥1.5 fold-change and false discovery rate (FDR) of 0.001 were used to define pathogenic processes. These included lymphocyte activation and associated extra-cellular matrix (ECM) responses (FIG. 1—Table). The master regulator of mitochondrial biogenesis PGC1A was suppressed four-fold in UC, in association with genes regulating mitochondrial biogenesis and ATP production. Remarkably, these same epithelial energy pathways were induced by 2′-FL supplementation in mice (FIG. 1—Table) (Mezoff et al., Am J Physiol Gastrointest Liver Physiol, 310:G427-38, 2016). Amongst moderate-to-severe patients treated with corticosteroids, week 4 remission rates were inversely correlated with the degree of dysregulation of the mitochondrial pathway, from 32% in the highest quartile for dysregulation to 71% in the lowest quartile (p=0.0004). These data indicate that approaches to boost rectal mitochondrial function, such as expansion of butyrate-producing microbiota by 2′-FL, could improve UC treatment responses. Collectively, these studies show that a gene signature for epithelial mitochondrial function induced by 2′-FL in mice is suppressed in association with shifts in butyrate-producing microbes and poor responses to therapy in pediatric CD and UC patients (Kugathasan et al. Lancet, 389:1710-1718, 2017; Mezoff et al., Am J Physiol Gastrointest Liver Physiol, 310:G427-38, 2016). These data provide support for 2′-FL supplementation in IBD, with enhancement of epithelial mitochondrial function via induction of butyrate-producing microbes as a novel therapeutic target.

### Risk/Benefit Assessment

### Known Potential Risks

The risks associated with this study are associated with 2′-FL or glucose supplementation and venipuncture. Risks associated with 2′-FL supplementation include potential dose-dependent increases in GI symptoms including: abdominal pain, nausea, loose stools, and/or gassiness. Risks associated with peripheral blood sampling (venipuncture) are: pain, bruising, fainting (rare), and/or infection (rare).

### Known Potential Benefits

Based on results of pre-clinical and clinical studies, dietary supplementation with the prebiotic 2′-FL can be effective for maintenance of IBD remission, for example, by boosting beneficial microbes, inhibiting harmful microbes, and suppressing pro-inflammatory cytokines. It has been shown to be safe and well tolerated in healthy infants and adults.

Results of this study provide valuable information regarding whether 2′-FL administration is safe and well tolerated in CD and UC patients in stable remission, and exerts a dose-dependent effect upon SCFA-producing microbiota which promote gut health and stable remission. This knowledge informs the design of a Phase III randomized clinical trial. Ultimately, this knowledge can be utilized to improve clinical practice.

### Assessment of Potential Risks and Benefits

The risks that participants are exposed to are likely mild to moderate. The study mitigates risks related to 2′-FL by having experienced gastroenterologists overseeing the study who are familiar with the profile of adverse reactions in this patient population. A dose range for 2′-FL which was well tolerated in a healthy adult population without changes in systemic (plasma cytokines) or mucosal inflammation (fecal calprotectin) is utilized. In addition, an adaptive dosing trial design is utilized so that participants randomized to the higher doses (5 g/d and 10 g/d doses) do not begin to take the drug until at least 10 participants have completed the lower dose.

Risks related to venipuncture are mitigated by having the procedure performed by expert personnel who are experienced in the care of pediatric patients.

The potential benefits of study participation outweigh the potential risks to participants.

Objectives and Endpoints

| | | JUSTIFICATION |
|---|---|---|
| OBJECTIVES | ENDPOINTS | FOR ENDPOINTS |
| Primary | | |
| Establish | SAFETY: | |
| the 2′-FL | GI symptoms (abdominal | Gastrointestinal |
| dosing and | pain, nausea, loose stools, | Symptom Rating |
| preliminary | gassiness) related to | Scale (GSRS) is a |
| safety and | tolerability collected using | validated symptom |
| efficacy data | the Gastrointestinal | scoring questionnaire |
| to guide design | Symptom Rating Scale (GSRS) | for patient-reported |
| of a phase III | | measures of |
| multi-center | | tolerability |
| study | EFFICACY: | |
| | Change in fecal | To assess if |
| | Bifidobacterium genus | supplementation |
| | abundance with 2′-FL or | with 2′-FL shifts |
| | glucose supplementation | the microbial |
| | within each dosing group | community towards |
| | between weeks 4 and 8. | greater numbers of |
| | | Bifidobacterium |
| Secondary | | |
| | SAFETY | |
| | Clinical relapse based on the | The wPCDAI and |
| | count of patients with an | PUCAI are validated |
| | increase of more than 20 | measures to collect |
| | points on the wPCDAI (CD | clinical disease |
| | patients) and 15 points for | activity in CD and |
| | the PUCAI (UC patients) | UC patients, |
| | between weeks 4 and 8. | respectively with |
| | | established cut |
| | | points for clinical |
| | | remission and relapse. |
| | Difference in mean change | |
| | across groups before and | |
| | after supplementation in the | |
| | following: | |
| | QoL IMPACT-III | IMPACT-III is a |
| | Plasma cytokines | validated |
| | Fecal calprotectin | Quality of Life |
| | | measure |
| | | Plasma cytokines |
| | | and fecal calprotectin |
| | | to assess systemic |
| | | and mucosal |
| | | inflammation |
| | EFFICACY: | |
| | Changes in fecal SCFA | To assess if 2′-FL |
| | including butyrate, pro- | supplementation |
| | inflammatory taxa including | shifts the microbial |
| | Enterobacteriaceae, plasma | community towards |
| | cytokines, and fecal | greater butyrate |
| | calprotectin with 2′-FL or | production, and |
| | glucose supplementation | reduces systemic and |
| | within each dosing group | mucosal inflammation |
| | between weeks 4 and 8. | as measured by |
| | | plasma cytokines and |
| | | fecal calprotectin, |
| | | respectively |
| Tertiary/ | | |
| Exploratory | | |
| Evaluate the | Age | Effects of these |
| effects of | Sex | variables are |
| biologic | Race | evaluated to guide |
| variables that | Fecal calprotectin at | the design of a |
| may influence | week 4 | multi-center RCT. |
| 2′-FL safety | Fecal microbiota at | |
| and efficacy | week 4 | |
| | Usual fiber intake | |
| | FUT2 secretor status | |

### Study Design

### Overall Design

This study evaluates whether 2′-FL supplementation in IBD is safe and well tolerated, while increasing fecal Bifidobacterium abundance and butyrate in a dose dependent manner. To do so, a two center Phase I/IIa double-blind, randomized, placebo-controlled dose-finding clinical trial is conducted. There are two study groups, one randomized to take 2′-FL and another randomized to a placebo group. Patients are randomized to placebo or treatment arm within strata using a staged approach, which allows for the assessment of safety for the lowest dosing group before randomization begins at the next highest dose. The potential for bias when randomizing patients to higher dosing groups at later time points is unlikely given the short duration of the trial and the inclusion of patients randomized to placebo and lower dosing groups at each stage allows for testing and accounting for any observed cohort/time effect.

Supplementation with 2′-FL or the glucose placebo takes place over a 4-week period. Daily doses are tracked and patient reported symptoms are captured weekly. The study requires 4 study visits: 1) a screening/baseline visit that includes a 4-week run-in period, 2) a visit for randomization, 3) a visit at the completion of dosing, and 4) a follow up visit 12 weeks later.

This study is the first dose-finding randomized clinical trial (RCT) of the 2′-FL prebiotic in IBD, utilizing state of the art metagenomic and metabolomic approaches to assess the response. Because a polymorphism in the FUT2 gene is associated with CD risk (McGovern et al., Hum Mol Genet, 19:3468-76, 2010), the optimal dose of 2′-FL in relation to FUT2 secretor status is also determined to inform future personalized clinical trials. These studies have a high impact in the field by providing critical phase I/IIa safety and efficacy data in support of a future phase III RCT to test the efficacy of 2′-FL in directly modulating beneficial microbiota and thereby enhancing sustained clinical remission. Ultimately these studies promote a fundamental shift in clinical practice towards personalized microbial therapeutic interventions.

### Dose

The recent dose finding RCT of 2′-FL in healthy adults found that it was safe and well tolerated at doses of 5, 10, and 20 g/d for two weeks (Elison et al., Br J Nutr, 116:1356-1368, 2016). A three-fold increase in Bifidobacterium, and a reduction in Proteobacteria, was observed with the 10 g dose (Elison et al., Br J Nutr, 116:1356-1368, 2016). A modest increase in GI symptoms including bloating and loose stools was reported at the 20 g dose, although no participant discontinued 2′-FL (Elison et al., Br J Nutr, 116:1356-1368, 2016). While a prior RCT in adults with CD demonstrated efficacy for reducing clinical disease activity and increasing butyrate production utilizing 20 g daily of a different prebiotic, oligofructose-enriched inulin, this was associated with a high rate of GI intolerance (primarily cramping and flatulence) and discontinuation of the study agent (De Preter et al., Clin Transl Gastroenterol, 18:1127-36, 2013). Therefore, in some instances a 20 g/d 2′-FL dose may not be well tolerated. In some instances a 10 g/d 2′-FL dose can be effective in increasing Bifidobacterium abundance and butyrate production. Therefore 1, 5, and 10 g/d of 2′-FL are assessed over 4 weeks in 20 UC and 20 CD subjects each. 2 g/d glucose are utilized as the placebo in 20 UC and 20 CD subjects.

It is possible that differences in the baseline microbial community and level of mucosal inflammation as measured by fecal calprotectin between CD and UC patients influence the response to 2′-FL supplementation. It is also possible that usual dietary fiber intake influences the response to 2′-FL (Holscher et al., J Nutr, 145:2025-32, 2015). A prior study of agave inulin prebiotic supplementation in healthy adults demonstrated a positive correlation between total dietary fiber+agave inulin intake and fecal butyrate (Holscher et al., J Nutr, 145:2025-32, 2015). Moreover, it was found in a study which included 142 pediatric IBD patients at three sites that mean(SD) dietary fiber intake was only 14(7) grams per day, at the low end of the range of consumption that was associated with appreciable fecal butyrate in healthy adults (Holscher et al., J Nutr, 145:2025-32, 2015). Moreover, GI intolerance of 2′-FL in IBD patients may occur at a lower dose than was detected in the recent RCT in healthy adults (Elison et al., Br J Nutr, 116:1356-1368, 2016; De Preter et al., Clin Transl Gastroenterol, 18:1127-36, 2013). Therefore, enrollment are balanced on disease phenotype (CD or UC) and usual dietary fiber intake and dosing group are randomized within strata of these factors.

### End of Study Definition

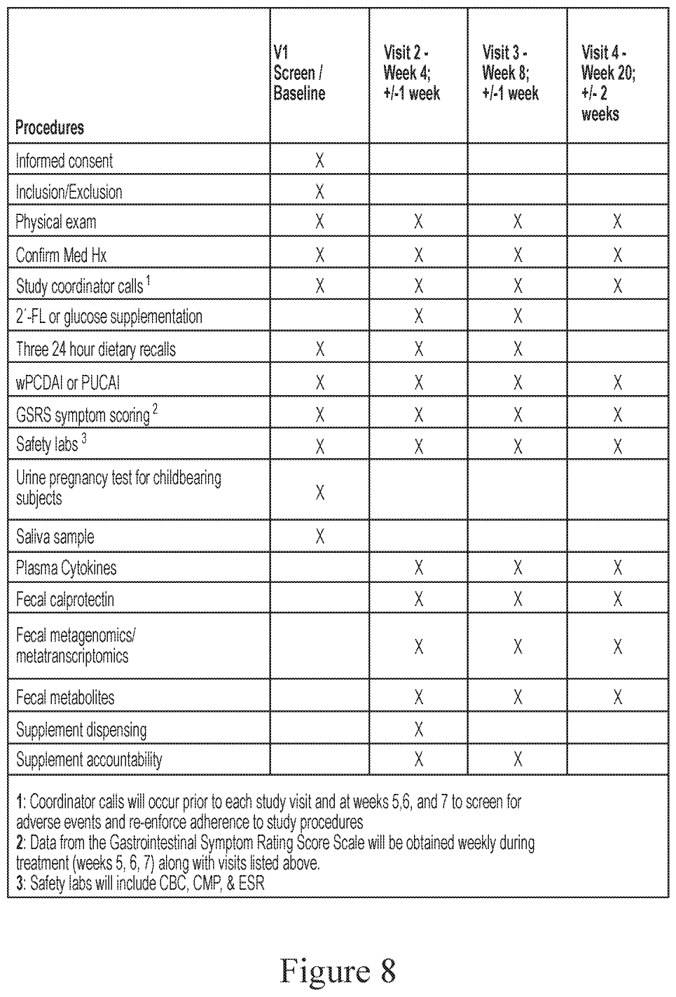

A participant is considered to have completed the study if he or she has completed all study visits including the last visit as shown in the Schedule of Activities (SoA) listed in FIG. 8.

### Study Population

### Inclusion Criteria

In order to be eligible to participate in this study, an individual must meet all of the following criteria: (1) Provision of signed and dated informed consent form; (2) Stated willingness to comply with all study procedures and availability for the duration of the study; (3) Male or female, aged 11-25; (4) Diagnosed with Crohn's Disease or Ulcerative Colitis; (5) Disease is in remission; (6) Not receiving corticosteroids; (7) Receiving a stable anti-TNF maintenance dose of adalimumab or infliximab; and (8) Agreement to not make any major dietary changes throughout study duration. This would include changing usual diet to a vegan diet, Specific Carbohydrate Diet (SCD), or exclusive enteral nutrition (EEN) diet.

### Exclusion Criteria

An individual who meets any of the following criteria are excluded from participation in this study: (1) Experienced a clinical relapse during the previous six months defined as wPCDAI or PUCAI>10; (2) Use of any of the following medications during the previous month: antibiotics, probiotics or prebiotics; (3) Diagnosis of celiac disease, diabetes or other co-morbidity that is determined as being exclusionary; (4) Treatment with another investigational drug or other intervention within 4 weeks; (5) Problem with lactose breakdown; (6) Pregnancy

Participants of childbearing potential are required to use an effective method of birth control while on study through at least 30 days after stopping the last dose of study supplement.

Participants who meet initial criteria undergo additional screening and a 4-week run-in period to collect baseline data. Participants who have any of the following additional exclusion criteria are excluded from further participation in the study.
Abnormal results of baseline screening labs defined as:
   WBC>1.5 ULN
   CMP w LFT>2.0 ULN
GI symptoms with a mean baseline total score >3 on the GSRS symptom questionnaire
wPDCAI and PUCAI values ≥10 at baseline and week 4

### Lifestyle Considerations

During this study, participants are asked to maintain a stable diet over the course of the study.

### Study Intervention Administration

### Study Intervention Description

2′-Fucosyllactose Powder (2′-FL) is a human milk oligosaccharide prebiotic. According to the FDA Guidance for Industry on Complementary and Alternative Medicine Products and Their Regulation by the Food and Drug Administration dated 2006, prebiotics are non-digestible food ingredients that affect the host beneficially by stimulating in a selective fashion the growth and/or activity of bacteria in the colon. According to the National Center for Complementary and Integrative Health (NCCIH), prebiotics are categorized as a biologically based practice. It is being used in this study as a complementary dietary supplement to anti-TNF therapy.

Glucose powder is utilized as the placebo comparator. Glucose is a primary source of energy and is naturally occurring in fruits and other parts of plants in its free state.

### Dosing and Administration

A total of 160 participants are randomized to consume one of three daily doses of 2′-FL or a glucose placebo for a period of 4 weeks. 80 participants are patients with Crohn's Disease and 80 participants are patients with Ulcerative Colitis. Participants are instructed that missed doses may be taken later on the day of the missed dose.

In the first arm, 120 participants receive 2′-FL in one of 3 groups:
Group 1: 1 g/d; n=40 (20 CD/20 UC)
Group 2: 5 g/d; n=40 (20 CD/20 UC)
   After 10 have completed Group 1
Group 3: 10 g/d; n=40 (20 CD/20 UC)
   After 10 have completed Group 2

In the second arm, 40 participants, 20 CD/20 UC receive glucose placebo at 2 g.

A stratified, staged randomization is employed. Strata is defined by study site, disease phenotype (e.g., CD/UC) and usual fiber intake (< or >=7 g/1000 kcal/day) resulting in eight equally balanced strata of 20 participants; this allows for assessment of safety for the lowest dosing group before randomization begins at the next highest dose. Should a dosing/disease phenotype group experience sufficient safety events, allocation to it and any higher dosing group, is terminated.

After the end of dosing at Week 8, participants complete a 12-week follow-up period to determine the stability of any changes detected for clinical disease activity, self-reported GI symptoms, plasma cytokines, and fecal calprotectin, microbiota, or metabolites during the period of supplementation.

### Formulation and Packaging

2′-Fucosyllactose (2′-FL) is a white homogenous powder and is neutral to slightly sweet with no off flavor. Dry matter makes up 96%, with a 4% moisture content. Results of analysis show that the overall content is: 2′-Fucosyllactose 93%, other sugars 3% and moisture 4%. The packaging consists of a multiple layered paper bag with a polyethylene liner and a volume of 25 kg net. The name of the agent appears on the label. Glucose is a white powder with a sweet taste.

Participants are instructed to take the 2′-FL or glucose each morning at breakfast by adding the required amount to a drink or food. Food diaries are kept by participants to record what food or drink the product was taken with.

### Measures to Minimize Bias: Randomization and Blinding

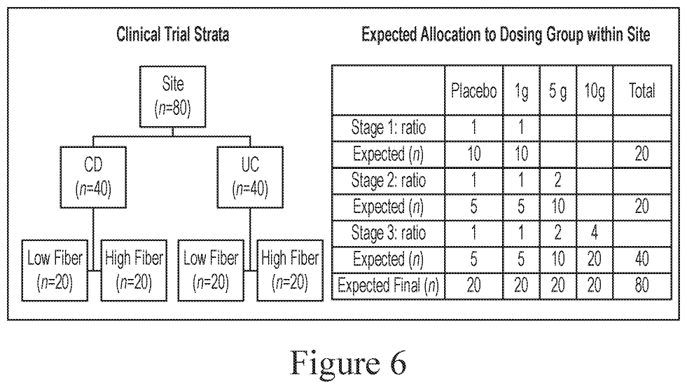

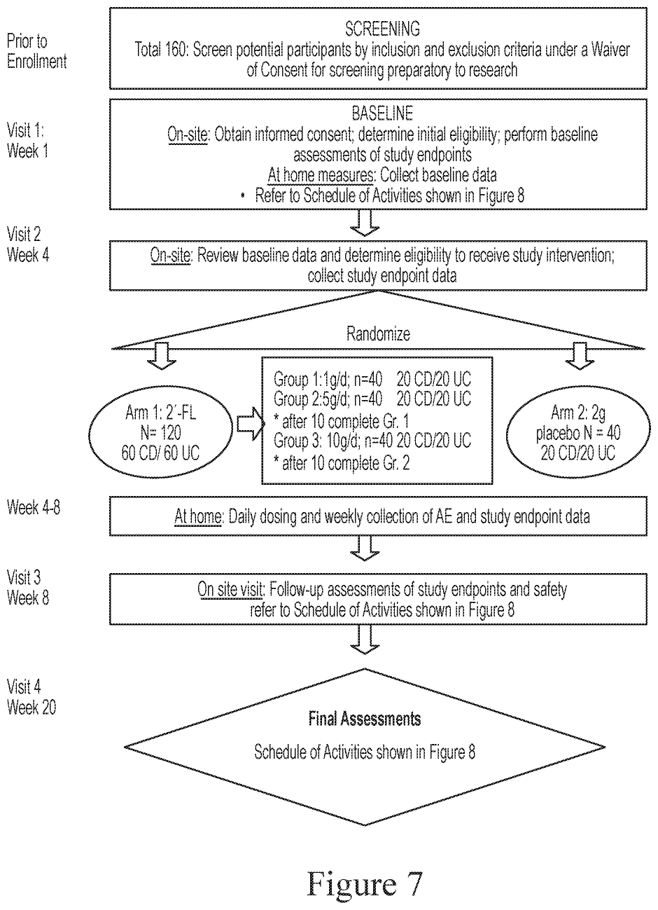

The overall enrollment and randomization schema is illustrated in FIG. 6. A double-blind placebo controlled, stratified, staged randomization is employed.

Strata is defined by study site, disease phenotype (e.g. CD/UC) and usual fiber intake (< or >=7 g/1000 kcal/day) resulting in eight equally balanced strata of 20 participants.

Patients are randomized to placebo or treatment arm within strata using a staged approach where in stage 1 randomization is a 1:1 ratio to placebo, 1 g; in stage 2 randomization 1:1:2 ratio to placebo, 1 g, 5 g; and in stage 3 randomization a 1:1:2:4 ratio to placebo, 1 g, 5 g, 10 g, resulting a total of 20 CD and 20 UC participants randomized to each dosing group. The sequential staging with shifting allocation ratio allows us to assess safety and tolerability for the lowest dosing group before randomization begins at the next highest dose. Should a dosing/disease phenotype group experience sufficient safety or intolerance events, allocation to it and any higher dosing group, are terminated. Natural block sizes of 2, 4, and 8 are used to randomize patients to dosing group within strata at stage 1, 2 and 3, respectively and an optimal randomization chosen at each stage to ensure balance.

### Study Intervention Compliance

Patient self-report data for 2′-FL intake using the Gastrointestinal Symptom Rating Scale is obtained at weeks 4, 5, 6, 7, and 8 when they are asked to record symptoms and daily 2′-FL consumption. Coordinators call participants weekly during the period of supplementation in an effort to improve 2′-FL adherence to acceptable levels, which are defined as consumption of the randomized dose on at least 24 out of 28 treatment days.

### Concomitant Therapy

Concomitant medications, including prescription medications, over-the-counter medications, and supplements are recorded at all study visits. Corticosteroids, antibiotics, probiotics, prebiotics (other than 2′-FL), and other investigational agents are not allowed during the study. In the event of a loss of clinical remission, participants are treated per standard of care.

### Study Assessments and Procedures

### Efficacy Assessments

The following assessments and procedures are conducted to obtain efficacy endpoint data:

### Stool Sample Collection and Sequencing:

Stool samples are collected from participants for DNA and RNA extraction. DNA and RNA are isolated with the AllPrep DNA/RNA Mini Kit (QIAGEN) with the addition of mechanical lysis. RNA is subsequently reverse transcribed into DNA and samples are quantified by Quant-iT PicoGreen dsDNA Assay (Life Technologies) and normalized to a concentration of 50 pg/ml. Whole-genome shotgun sequencing libraries are prepared according to the manufacturer's instructions using the Nextera XT DNA Library Preparation kit (Illumina) with 100-250 pg input DNA. Libraries are pooled by transferring equal volumes of each library using a Labcyte Echo 550 liquid handler. The concentrations and insert size ranges for each pooled library are checked using an Agilent Bioanalyzer DNA 1000 kit (Agilent Technologies). Libraries are subsequently sequenced on the Illumina HiSeq 2000 platform in paired-end mode (2×101 bp) targeting 2.5 Gb of sequences per sample (Kugathasan et al. Lancet, 389:1710-1718, 2017; Schirmer et al., Cell, 167:1125-1136 e8, 2016).

### Metagenomic and Metatranscriptomic Analysis:

Samples are included in the analysis if they had sufficient sequencing reads. Reads were first processed using KneadData (huttenhower.sph.harvard.edu/kneaddata). This included quality-trimming (trimmomatic parameters: MAXINFO:90:0.5), read-filtering based on a minimum read length of 60 bp, and removal of potential human contamination by filtering reads that aligned to the human genome (reference genome hg19). Quality-controlled, paired-end reads were aligned against a database of unique clade-specific marker genes using Bowtie2 and taxonomic profiles were inferred with MetaPhlAn 2.2 (Segata et al., 2012). For subsequent analysis, species as well as genus composition of the samples were considered. Functional profiling was performed using HUMAnN2 (huttenhower .sph.harvard.edu/humann2). Briefly, reads are mapped against a customized database of functionally annotated pangenomes, only considering organisms that were identified during the taxonomic profiling step. Functional annotation of the protein sequences in the pangenomes to their respective UniRef50 family is provided with the software. Reads that cannot be mapped are subsequently aligned against the complete UniRef50 database. The community totals are computed for each protein family (RPK) and converted into relative abundances. For subsequent downstream analysis, these tens of thousands of gene families were further grouped into broader functional categories: MetaCyc metabolic pathways and informative GO categories, focusing on molecular functions and biological processes.

Specifically, the selected GO terms were each annotated to >2,000 proteins in UniRef50, while all their descendant (more specific) terms were annotated to <2,000 proteins.

### Metabolomic Analysis:

Data is acquired using LC-MS systems comprised of Nexera X2 U-HPLC systems (Shimadzu Scientific Instruments; Marlborough, Mass.) and Q Exactive/Exactive Plus orbitrap mass spectrometers (Thermo Fisher Scientific; Waltham, Mass.) using negative ion mode MS analysis of polar metabolites. LC-MS samples are prepared from stool homogenates (30 μL) via protein precipitation with the addition of four volumes of 80% methanol containing inosine-N4 (Rios-Covian et al., FEMS Microbial Lett; 2015), thymine-d4 and glycocholate-d4 internal standards (Cambridge Isotope Laboratories; Andover, Mass.). The samples are centrifuged (10 min, 9,000×g, 4° C.) and the supernatants are injected directly onto a 150×2.0 mm Luna NH2 column (Phenomenex; Torrance, Calif.). The column is eluted at a flow rate of 400 μL/min with initial conditions of 10% mobile phase A (20 mM ammonium acetate and 20 mM ammonium hydroxide in water) and 90% mobile phase B (10 mM ammonium hydroxide in 75:25 v/v acetonitrile/methanol) followed by a 10 min linear gradient to 100% mobile phase A. MS analyses are carried out using electrospray ionization in the negative ion mode using full scan analysis over m/z 60-750 at 70,000 resolution and 3 Hz data acquisition rate. Additional MS settings are: ion spray voltage, −3.0 kV; capillary temperature, 350° C.; probe heater temperature, 325° C.; sheath gas, 55; auxiliary gas, 10; and S-lens RF level 40.

### Data Processing:

Raw LC-MS data are acquired to the data acquisition computer interfaced to each LC-MS system and then stored on a robust and redundant file storage system (lsilon Systems) accessed via the internal network at the Broad Institute. Data processing is conducted using one of five Dell Precision T7600 workstations, each equipped with eight core XEON E5-2687W processors, 32 GB of DDR3 RAM, and 2 TB of storage in RAID 0 array of four 600 GB SAS hard drives. Nontargeted data are processed using Progenesis CoMet software (v 2.0, Nonlinear Dynamics) to detect and de-isotope peaks, perform chromatographic retention time alignment, and integrate peak areas. Peaks of unknown ID are tracked by method, m/z and retention time. Identification of nontargeted metabolite LC-MS peaks is initially conducted by i) matching measured retention times and a masses to mixtures of references metabolites analyzed in each batch, ii) matching an internal database of >600 compounds that have been characterized using the Broad Institute methods, and iii) matching exact masses only to an external database of >40000 metabolites (Human Metabolome Database v3) (Wishart et al., Nucleic Acids Res, 41:D801-7, 2013). Compounds matched to the external database are confirmed by analyzing reference standards if they are available.

### Demographics:

Information related to participant age and sex is collected as part of the review of medical records conducted for screening (see below). Race is collected at Visit 1.

### Fecal Calprotectin:

Fecal calprotectin is measured using a monoclonal antibody-based ELISA which has demonstrated superior linearity over a wide dynamic range (Bohlmann Laboratories, Switzerland) (Burri, et al. Clin Chim Acta, 416:41-7, 2013).

### Plasma Cytokines:

Thirteen plasma cytokines representing innate and adaptive immune responses are measured using a high sensitivity bead-based multiplex assay as previously reported (Milliplex Multiplex Assay using Luminex) (Dorn et al., Psychosom Med, 78:646-56, 2016).

### Fiber Intake:

Three unannounced 24-hour dietary recall interviews are administered at baseline and weeks 4 and 8 to allow for randomization of patients within strata (high/low) of usual fiber intake and determination of whether differences in usual diet are associated with differential responses to 2′FL. The dietary recall is performed by an expert interviewer using the USDA's Automated Multiple Pass Method (AMPM) to ensure accurate and consistent capture of foods and amounts reported by the participant (Moshfegh et al., Am J Clin Nutr, 88:324-32, 2008). Nutrition Data Systems for Research (NDSR) (Nutrition Coordinating Center, University of Minnesota, Minneapolis, Minn.) software and foods database are used to assess total daily energy, macronutrient, and fiber intake, as well as food group servings consumed (Sievert et al., Control Clin Trials, 10:416-25, 1989).

### FUT2 Secretor Status:

FUT2 secretor status has been implicated in both infectious and inflammatory conditions, and in opposing directions. FUT2+(secretor) individuals experience increased risk of rotavirus and norovirus gastroenteritis (Currier et al., Clin Infect Dis, 60:1631-8, 2015; Payne et al., JAMA Pediatr, 169:1040-5, 2015), whereas FUT2-(non-secretor) individuals experience increased risk of CD (McGovern et al., Hum Mol Genet, 19:3468-76, 2010). Moreover, FUT2 non-secretors may exhibit reductions in 2′-FL target microbiota including Bifidobacterium even in the absence of mucosal inflammation (Rausch et al., Proc Natl Acad Sci USA, 108:19030-5, 2011; Tong et al., ISME J, 8:2193-206, 2014; Wacklin et al., PLoS One; 2011, 6:e20113, Wacklin et al., PLoS One, 9:e94863, 2014). Secretor status can be measured by genotype or phenotype. Genotyping in the U.S. involves analysis of a single nucleotide 428G>A polymorphism in the FUT2 gene (rs601338). 23% of the U.S. population is homozygous for this inactivating mutation, which results in deficiency of fucosylated gut carbohydrate. Phenotypically, non-secretor status can be measured by testing a whole saliva sample for “secretor carbohydrate” using the Ulex europaeus-1 (UEA-1) lectin enzyme immunoassay. The UEA-1 immunoassay detects alpha1,2-fucose-linked products of the FUT2 gene enzyme (Kazi et al., J Infect Dis, 215:786-789, 2017; Morrow et al., J Pediatr, 158:745-51, 2011). Studies have found that some FUT2+secretor individuals—who are genetically capable of synthesizing secretor carbohydrate—produce low quantity of secretor carbohydrate, and appear phenotypically similar to non-secretor individuals. Therefore in this study, both FUT2 genotype and phenotype are measured.

### Safety and Other Assessments

Prior to enrollment the following are performed for screening purposes.

### Review of Medical Records:

Existing information about potential participants are reviewed to determine sex, age, diagnosis, current medications, medication history, co-morbidities, and allergies.

The following assessments and procedures are performed to determine eligibility for study participation (Patient Population) and throughout the study to obtain safety data:

### Physical Exam and Vital Signs:

A physical exam is conducted at each study visit. Vital signs are collected and include temperature, heart rate, respiratory rate, and blood pressure. Weights are also collected at these visits.

### Blood Collection:

A blood sample is drawn and analyzed for CBC, CMP, & ESR

### Urine Collection:

Female participants who are capable of becoming pregnant have a urine pregnancy test at baseline visit.

### Saliva Sample:

A saliva sample is collected at Visit 1 to measure FUT2 phenotype secretor status.

Weighted Pediatric Crohn's Disease Activity Index (wPCDAI) and Pediatric Ulcerative Colitis Activity Index (PUCAI):

The wPCDAI and the PUCAI are utilized to measure clinical disease activity in the CD and UC, groups, respectively. These have been validated in the pediatric IBD population with well-established cut-points for clinical remission and relapse. wPCDAI and PUCAI scores are obtained at baseline and weeks 4, 12, and 20 (Turner et al., Inflamm Bower Dis, 15:1218-23, 2009; Turner et al., Gastroenterology, 133:423-32, 2007). For both wPCDAI and PUCAI, values <10 are required at baseline and week 4 to meet entry criteria for stable clinical remission.

IMPACT III: the IMPACT-III questionnaire are used to measure quality of life (QOL) at baseline, and weeks 4, 8, and 20. IMPACT-III has been validated in the IBD population with excellent reliability for the total score (Otley et al., J Pediatr Gastroenterol Nutr, 35:557-63, 2002; Otley et al., Inflamm Bowel Dis, 12:684-91, 2006). A score of 144 or greater is used as indicative of a good quality of life.

### GSRS Questionnaire:

The GSRS questionnaire is utilized to track patient-reported measures of 2′-FL tolerability including abdominal pain, nausea, loose stools, and gassiness. These parameters were modestly increased in healthy adults who received the 20-g dose of 2′-FL in the recent RCT, but did not vary at lower doses of 2′-FL. Each participant is provided the GSRS questionnaire at each study visit. Then, each participant collects the GSRS questionnaire on a weekly basis during the treatment (week 4 to 8) phase. The severity of 15 gastrointestinal symptoms is reported on a seven-point Likert scale ranging from (1) no symptoms to (7) severe symptoms, and an average score is computed for each participant for the baseline to week 4, week 4 to week 8, and week 8 to week 20 time periods.

### Usual Diet Including Daily Dietary Fiber Intake:

Three unannounced 24-hour dietary recall interviews are administered at baseline and weeks 4 and 8 to allow for the randomization of patients within strata (high/low) of usual fiber intake and determination of whether differences in usual diet are associated with differential responses to 2′FL. The dietary recall is performed by an expert interviewer using the USDA's Automated Multiple Pass Method (AMPM) to ensure accurate and consistent capture of foods and amounts reported by the participant (Moshfegh et al., Am J Clin Nutr, 88:324-32, 2008). Nutrition Data Systems for Research (NDSR) (Nutrition Coordinating Center, University of Minnesota, Minneapolis, Minn.) software and foods database are used to assess total daily energy, macronutrient, and fiber intake, as well as food group servings consumed (Sievert et al., Control Clin Trials, 10:416-25, 1989). Patients are encouraged to maintain a stable diet over the course of the study.

### Plasma Cytokines and Fecal Calprotectin:

Plasma cytokines and fecal calprotectin are measured to assess systemic and mucosal inflammation, respectively. Thirteen plasma cytokines representing innate and adaptive immune responses are measured using a high sensitivity bead-based multiplex assay. Fecal calprotectin are measured using a monoclonal antibody-based ELISA which has demonstrated superior linearity over a wider dynamic range than other available assay kits.

### Statistical Considerations

Aim 1 studies determine whether 2′-FL administration is safe and well tolerated in CD and UC patients in stable remission receiving maintenance anti-TNF therapy. Aim 2 studies focuses on measures of 2′-FL efficacy in shifting the microbial community towards greater Bifidobacterium abundance and butyrate production, and reducing systemic and mucosal inflammation as measured by plasma cytokines and fecal calprotectin, respectively. This includes fecal microbial metagenomics, metatranscriptomics, and metabolomics. These are tested at weeks 4, 8, and 20. The same methodology are employed as for the current PROTECT, RISK, and HMP2 studies (Integrative HMPRNC, Cell Host Microbe, 16:276-89, 2014).

Aim 1: Define the dose dependent safety and tolerability of 2′-FL as a dietary supplement in IBD. It is expected that 2′-FL is safe and well tolerated as a dietary supplement in IBD patients in remission.
Primary Aim 1 Endpoint: Change in the GSRS symptom score for tolerability with 2′-FL or glucose supplementation within each dosing group between weeks 4 and 8.
Secondary Aim 1 Endpoint(s): Clinical relapse using the wPCDAI for CD patients and the PUCAI for UC patients, plasma cytokines, and fecal calprotectin.
Aim 2: Define the dose dependent efficacy of 2′-FL as a dietary supplement in IBD. It is expected that 2′-FL increases fecal Bifidobacterium abundance and butyrate in a dose dependent manner.
Primary Aim 2 Endpoint: Change in fecal Bifidobacterium genus abundance with 2′-FL or glucose supplementation within each dosing group between weeks 4 and 8.
Secondary Aim 2 Endpoints: Changes in fecal SCFA including butyrate, pro-inflammatory taxa including Enterobacteriaceae, plasma cytokines, and fecal calprotectin with 2′-FL or glucose supplementation within each dosing group between weeks 4 and 8.

### Sample Size Determination

The sample size of 20 participants per 2′-FL dosing group within CD or UC is based on the primary efficacy endpoint, the increase in fecal Bifidobacterium, as described under Aim 2. The primary end point for Aim 1 is the mean change in the GSRS tolerability score in each of the 2′-FL dosing groups and the glucose placebo group. In the recent 2′-FL RCT in healthy adults, 10 participants per dosing group were sufficient to demonstrate an increase in mild GI symptoms in the 20 g 2′-FL group compared to the 2 g glucose placebo group. This included an increase in the mean(SD) daily frequency of bowel movements from 1.3(0.3) to 1.6(0.4) in the 20 g 2′-FL group. With 20 CD or UC participants per 2′-FL dosing group, and assuming a baseline total GSRS score of 2.5 and no change with placebo, a difference across dosing groups should be detected for a linear change in the total GSRS score in as small as 0.2 units for 1 g, 0.7 for 5 g, and 1.25 for 10 g 2′-FL at a=0.05 (two-sided) and power=0.80. In addition, the statistical power is sufficient for detection of a two-fold increase in total GSRS score should be able to be detected after the first 10 subjects are randomized to placebo and 1 g 2′-FL should the mean difference/standard deviation (i.e. standardized effect size) not exceed 1.25. For example, the statistical power should be sufficient to detect a difference of 2.5 in total GSRS score between placebo and 1 g 2′-FL if the pooled standard deviation does not exceed SD=2. Greater power is realized at stage 2 given the larger number randomized to placebo. A 20% drop-out or not evaluable rate is assumed, and so 100 CD and 100 UC participants are enrolled.

Examination of shotgun metagenomic sequence data from pediatric and young adult CD patients receiving infliximab and with fecal calprotectin levels <250 μg/g indicates that baseline variability in Bifidobacterium abundance in this study may be as high as SD=10.6% (Lewis et al., Cell Host Microbe, 18:489-500, 2015). The sample size determination was performed on the following assumptions: baseline mean Bifidobacterium abundance of 6.8%; an expected linear dose response in mean Bifidobacterium abundance of 6.8% for placebo, 7.6% for 1 g, 10.7% for 5 g and 14.6% for 10 g 2-FL; a pooled standard deviation in Bifidobacterium abundance of 10.6%; and equal allocation to dosing groups. Thus, for a=0.05 (two-sided) and power=0.80, a total of 20 patients in each group allows for detection of a mean difference in the response to intervention at levels corresponding to those reported for adults. Should the variation in Bifidobacterium abundance be closer to that observed in healthy adults greater power is realized.

### Consideration of Relevant Biologic Variables.

Biologic variables which may influence 2′-FL safety and efficacy include age, sex, race/ethnicity, FUT2 secretor status, IBD diagnosis of CD or UC, dietary fiber intake, mucosal inflammation as measured by fecal calprotectin, and the baseline microbial community (Lewis et al., Cell Host Microbe, 18:489-500, 2015, Currier et al., Clin Infect Dis, 60:1631-8, 2015; Tong et al., ISME J, 8:2193-206, 2014; Wacklin et al., PLoS One, 6:e20113, 2011). Of these, IBD diagnosis of CD or UC and dietary fiber intake are likely to have the greatest effect. Therefore, sufficient participants with CD or UC are enrolled in each dosing group to evaluate these independently, and enrollment are balanced across the four groups for usual dietary fiber intake. Equal numbers of males and females ages 11 and above, and Caucasian (90%) and African-American (10%) subjects in proportion to the overall IBD populations are enrolled.

### Populations for Analyses

The primary analysis are on a per protocol basis, including only patients who consumed at least 24 out of 30 2′-FL doses to which they were randomized (Elison et al., Br J Nutr, 116:1356-1368, 2016). The secondary analysis are based on an Intent to Treat (ITT) schema, with each patient included in the group to which they were randomized.

### Statistical Analyses

### General Approach

Descriptive statistics and graphical analyses are used to describe GSRS tolerability scores, clinical relapse rates, disease activity index scores, plasma cytokines, fecal calprotectin, and QoL across the four groups at each time point.

### Analysis of Primary Aim Endpoints

The primary safety outcome utilizes descriptive statistics and graphical analyses are used to describe clinical relapse rates, disease activity index scores, plasma cytokines, fecal calprotectin, and tolerability scores for abdominal pain, nausea, loose stools, and gassiness, across the four groups at each time point. The primary determination of tolerability are based on the Gastrointestinal Symptom Rating Scale (GSRS), the same measure for tolerability utilized in the recent 2′-FL RCT in healthy adults (Elison et al., Br J Nutr, 116:1356-1368, 2016). The rate of clinical relapse, and change in the GSRS, within the glucose placebo group between weeks 4 and 8 are utilized to assess safety and tolerability of each dose of 2′-FL. If a two-fold increase in the GSRS in a CD or UC 2′-FL dosing group is observed compared to the glucose placebo group, it is concluded that that dose was not well tolerated. If two more subjects in a CD or UC 2′-FL dosing group experience clinical relapse, in excess of the rate of clinical relapse observed in the glucose placebo group, it is concluded that the dose was not safe.

The primary efficacy outcome is the difference in mean change across dosing groups in fecal Bifidobacterium abundance before and after supplementation. The difference in Bifidobacterium abundance is examined using linear mixed-effects regression with the time-by-treatment interaction term providing the test for mean change as described under Aim 1. Post hoc tests for differences across specific dosing groups are compared using linear contrasts with a focus on identifying a linear trend for increasing 2-FL dose. Tests are conducted separately for CD and UC patients. Ordinations and statistical learning approaches for high-dimensional data are used to identify differences in microbial community structure in response to 2′-FL supplementation.

### Analysis of Secondary Aim Endpoints

Secondary safety and tolerability outcomes examine the difference in mean change across dosing groups in disease activity index scores, plasma cytokines, fecal calprotectin, tolerability scores, and QoL before and after supplementation. Differences are examined using linear mixed-effects regression (LMER) with the time-by-treatment interaction term providing the test for mean change. Post hoc tests for differences across specific dosing groups are compared using linear contrasts with a focus on identifying whether safety and tolerability is impacted at higher dosing levels. Tests are conducted separately for CD and UC patients to assess differential response to treatment by disease phenotype. Formal tests for interaction are conducted should appreciable differences be observed. Safety and tolerability measures collected at week 12 are incorporated into the LMER framework to examine the stability of symptoms at follow-up. The LMER framework are also used to test for differences in the weekly rate of change in tolerability from the GSRS by nesting observations within subjects and testing for differences in the slopes according to dosing group. Potential non-linear associations with time are identified using graphical approaches and model fit statistics and modeled using polynomial terms or restricted cubic splines as appropriate. Differences in week 8 clinical relapse rates between each of the 2′-FL intervention groups are compared using Fisher's exact test. Within-dose comparisons for the number of relapses between weeks 4 and 8 are conducted using exact test for paired data. Based upon the recent 2′-FL RCT in healthy adults, it is anticipated that each of the three doses of 2′-FL are safe and well tolerated compared to the glucose placebo in both the CD and UC groups. It is expected that FUT2 secretors and non-secretors exhibit similar profiles for safety and tolerance for each of the 2′-FL doses.

LMER are also used to test for mean differences in secondary efficacy outcomes including fecal calprotectin, GI symptom tolerability score, plasma cytokines and Enterobacteriaceae before and after supplementation. Based upon the recent RCT in healthy adults, it is expected to detect a two-fold increase in fecal Bifidobacterium abundance, and a significant reduction of taxa within the Proteobacteria phylum including Enterobactericae, at week 8 following supplementation with 2′-FL at the 10 g dose. A recent pediatric CD study demonstrated no change in overall mean(SD) fecal Bifidobacterium abundance over 8 weeks of anti-TNF induction therapy, despite a reduction in mucosal inflammation as measured by fecal calprotectin (baseline: 6.5(14.8) vs. week 8: 6.8 (10.6)). Therefore, it is expected that there is no change in fecal Bifidobacterium abundance with glucose supplementation, showing specificity of the 2′-FL response, and lesser changes with the 1 g and 5 g 2′-FL doses, demonstrating dose dependency. Consistent with the increase in fecal SCFA-producing microbes, it is expected that the SFCA analysis detects a significance increase in fecal butyrate concentration with 2′-FL supplementation. As 2′-FL supplementation of infant formula has been shown to reduce circulating plasma cytokines in healthy neonates, it is expected that that 2′-FL supplementation also reduces both plasma cytokines and fecal calprotectin in a dose dependent manner, with no change with glucose supplementation. It is expected that FUT2 non-secretors exhibit a trend towards a greater benefit of 2′-FL supplementation.

### Safety Analyses

Based upon data from the IBD patient population management report which is collected for the ImproveCareNow (ICN) Quality Improvement Collaborative, it is expected that not more than 10% of patients receiving stable maintenance anti-TNF therapy are expected to experience a clinical relapse over a four-week period (Minar et al., Inflamm Bowel Dis, 22:2641-2647, 2016; Minar et al., J Pediatr Gastroenterol Nutr, 62:715-22, 2016). For each of the three doses of 2-′FL and the glucose placebo the number of subjects who experience clinical relapse are determined. The rate of clinical relapse, and change in the GI symptoms tolerability score collected using the GSRS questionnaire, within the glucose placebo group for CD or UC are utilized to assess safety and tolerability of each dose of 2′-FL. If two more subjects in a CD or UC dosing group experience clinical relapse, in excess of the rate of clinical relapse observed in the placebo group, it is concluded that that dose was not safe. Similarly, if a significant two-fold increase in the GI symptoms tolerability score collected using the GSRS questionnaire, is observed compared to the placebo group, in a CD or UC dosing group experience it is concluded that that dose was not well tolerated.

### Baseline Descriptive Statistics

Descriptive statistics and graphical analyses are used to describe clinical and demographic characteristics, FUT2 secretor status and dietary fiber intake, and baseline GSRS tolerability scores, plasma cytokines, fecal calprotectin, fecal microbial community and functions, and Qol across the four groups at study entry.

### Planned Interim Analyses

Interim analyses assess the safety and tolerability of each 2′-FL dose prior to randomization of participants to the next highest dose. Patients are randomized to placebo or treatment arm within strata using a staged approach where in stage 1 randomization is a 1:1 ratio to placebo or 1 g; stage 2 a 1:1:2 ratio to placebo, 1 g, 5 g; and stage 3 a 1:1:2:4 ratio to placebo, 1 g, 5 g, 10 g, resulting an expected total of 20 CD and 20 UC participants randomized to each dosing group. The sequential staging with shifting allocation ratio allow for the assessment of safety and tolerability for the lowest dosing group before randomization begins at the next highest dose. Should a dosing/disease phenotype group experience sufficient safety or intolerance events, allocation to it and any higher dosing group, is terminated. Natural block sizes of 2, 4, and 8 are used to randomize patients to dosing group within strata at stage 1, 2 and 3, respectively and an optimal randomization chosen at each stage to ensure balance. An independent statistician generates the randomization and provides the computer-generated lists to the pharmacy for dispensing. Advantages of this approach are that it allows for the assessment of safety before moving to a higher dose and balance across factors with the potential to influence response to treatment. The potential for bias when randomizing patients to higher dosing groups at later time points is unlikely given the short duration of the trial. The inclusion of patients randomized to placebo and lower dosing groups at each stage allow for testing and accounting for any observed cohort/time effect.

### Sub-Group Analyses

The primary Aim 1 analysis is on a per protocol basis, including only patients who consumed at least 24 out of 28 2′-FL doses to which they were randomized (Elison et al., Br J Nutr, 116:1356-1368, 2016). The secondary analysis is based on an Intent to Treat (ITT) schema, with each patient included in the group to which they were randomized. Descriptive statistics and graphical analyses are used to describe GSRS tolerability scores, clinical relapse rates, disease activity index scores, plasma cytokines, fecal calprotectin, and Qol across the four groups at each time point. Safety and tolerability outcomes examine the difference in mean change across dosing groups in the GSRS, disease activity index scores, plasma cytokines, fecal calprotectin, and Qol before and after supplementation. Clinical relapse are defined as an increase of 20 or more points for the wPCDAI, and 15 or more points for the PUCAI, between weeks 4 and 8 (Haberman et al., J Clin Invest, 124:3617-33, 2014; Holscher et al., J Nutr; 2015, 145:2025-32, Schirmer et al., Cell, 167:1125-1136 e8, 2016; Wishart et al., Nucleic Acids Res, 41:D801-7, 2013). Key patient-reported outcome (PRO) components of pain and stools for CD, and stools and blood for UC are also separately examined. The primary measure of tolerability is the mean change across dosing groups in the GSRS. Differences are examined using linear mixed-effects regression (LMER) with the time-by-treatment interaction term providing the test for mean change. For primary analyses testing changes from weeks 4 to 8 (i.e. intervention pre-posttest) the Kenward-Roger correction is used to obtain the correct degrees of freedom for the F-tests and an unstructured correlation structure specified. Post hoc tests for differences across specific dosing groups are compared using linear contrasts with a focus on identifying whether safety and tolerability is impacted at higher dosing levels. Tests are conducted separately for CD and UC patients to assess differential response to treatment by disease phenotype. Formal tests for interaction are conducted should appreciable differences be observed. Safety and tolerability measures collected at week 12 are incorporated into the LMER framework to examine the stability of symptoms at follow-up. The LMER framework are also used to test for differences in the weekly rate of change in GSRS tolerability scores by nesting observations within subjects and testing for differences in the slopes according to dosing group. Potential non-linear associations with time are identified using graphical approaches and model fit statistics and modeled using polynomial terms or restricted cubic splines as appropriate. Differences in week 8 clinical relapse rates between each of the 2′-FL intervention groups are compared using Fisher's exact test. Within-dose comparisons for the number of relapses between weeks 4 and 8 are conducted using the exact test for paired data. Based upon the recent 2′-FL RCT in healthy adults, it is expected that each of the three doses of 2′-FL are safe and well tolerated compared to the glucose placebo in both the CD and UC groups. This is determined by demonstrating no difference for the mean change in the GSRS tolerability score, or rates of clinical relapse, between each of the 2′-FL dosing groups and the glucose placebo group.

The primary Aim 2 analysis is on a per protocol basis, including only patients who completed all of the study procedures including at least 24 out of 28 doses of the supplementation to which they were randomized. The secondary analysis is based on an Intent to Treat (ITT) schema, with each patient included in the group to which they were randomized. Descriptive statistics are used to present differences in microbiota taxonomic and functional profiles, fecal SCFA, plasma cytokines, and fecal calprotectin across the four groups. The primary efficacy outcome is the difference in mean change across dosing groups in fecal Bifidobacterium abundance before and after supplementation. The difference in Bifidobacterium abundance is examined using linear mixed-effects regression (LMER) with the time-by-treatment interaction term providing the test for mean change as described under Aim 1. Post hoc tests for differences across specific dosing groups are compared using linear contrasts with a focus on identifying a linear trend for increasing 2-FL dose. In addition to comparing the relative abundance of Bifidobacterium across different dosage groups, differences in within-patient variation in Bifidobacterium are also assessed using Flinger-Killeen test of homogeneity of variances. These relative changes are subsequently compared to changes in absolute abundance measurements via qPCR. Tests are conducted separately for CD and UC patients. Furthermore, differences in overall community composition are explored using Principal Coordinate Analysis with Bray-Curtis distance. This is followed by the identification of specific taxonomic and functional microbial features that are associated with the response to 2′-FL supplementation. To this end, a linear modeling system adapted for microbial community data is used and control for the major factors impacting the microbiome such as age, ethnicity and gender and account for multiple samples from the same patient (MaAslin: huttenhower.sph.harvard.edu/maaslin). In addition, strain-level differences in the patient groups are examined by comparing SNP profiles of species with sufficient coverage and their functional implications. LMER is also used to test for mean differences in secondary efficacy outcomes including fecal calprotectin, GSRS tolerability score, plasma cytokines, SCFA, and Enterobacteriaceae before and after supplementation. Based upon the recent RCT in healthy adults, it is expected to detect a two-fold increase in fecal Bifidobacterium abundance, and a significant reduction of taxa within the Proteobacteria phylum including Enterobactericae, at week 8 following supplementation with 2′-FL at the 10 g dose. A recent pediatric CD study demonstrated no change in fecal Bifidobacterium abundance over 8 weeks of anti-TNF induction therapy, despite a reduction in mucosal inflammation as measured by fecal calprotectin (Lewis et al., Cell Host Microbe, 18:489-500, 2015). Therefore, a smaller degree of variation in fecal Bifidobacterium abundance is expected in patients with glucose supplementation in contrast to patients with 2′-FL supplementation, indicating specificity of the 2′-FL response, and lesser changes with the 1 g and 5 g 2′-FL doses, demonstrating dose dependency. It is expected that the metabolite analysis detects a significance increase in fecal butyrate concentration with 10 g 2′-FL supplementation. As 2′-FL supplementation of infant formula has recently been shown to reduce circulating plasma cytokines in healthy neonates, it is expected that 2′-FL supplementation also reduces both plasma cytokines and fecal calprotectin in a dose dependent manner, with no change with glucose supplementation (Goehring et al., J Nutr, 146:2559-2566, 2016). This is followed by Hierarchical All-against-All significance testing (HAllA: huttenhower.sph.harvard.edu/halla) to identify groups of bacterial species and functions that are associated with changes in SCFA, fecal calprotectin and plasma cytokines. It is expected that FUT2 non-secretors exhibit a trend towards a greater benefit of 2′-FL supplementation.

### Exploratory Analyses

Effects of age, sex, race, week 4 fecal calprotectin and microbiota, usual fiber intake, and FUT2 secretor status are tested in an exploratory manner to guide design of the multi-center RCT. Descriptive statistics are calculated and models fit separately for each of these subgroups with formal tests of interaction considered should material differences be observed.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method for alleviating or reducing the risk of relapse in inflammatory bowel disease (IBD), the method comprising administering to a subject in need thereof an effective amount of a 2′-fucosyllactose compound, wherein the subject is a human IBD patient who has undergone or is on an anti-inflammatory therapy.

2. The method of claim 1, wherein the human patient is in remission of the IBD.

3. The method of claim 1, wherein human patient is receiving the anti-inflammatory therapy and wherein the 2′-fucosyllactose compound is administered as an adjunct to the anti-inflammatory therapy.

4. The method of claim 1, wherein the anti-inflammatory therapy is an anti-TNF therapy.

5. The method of claim 4, wherein the anti-TNF therapy comprises infliximab and/or adalimumab.

6. The method of claim 1, wherein the 2′-fucosyllactose compound is administered to the human patient in an amount sufficient to increase abundance of intestinal microbes that produce short-chain fatty acids in the human patient.

7. The method of claim 6, wherein the intestinal microbes comprise Bifidobacteria, Bacteroides, and/or Parabacteroides.

8. The method of claim 1, wherein the 2′-fucosyllactose compound is administered to the human patient in an amount sufficient to decrease intestinal calprotectin of the human patient.

9. The method of claim 1, wherein the 2′-fucosyllactose compound is formulated in a composition comprising the 2′-fucosyllactose compound as the only oligosaccharide content.

10. The method of claim 1, wherein the 2′-fucosyllactose compound is formulated in a composition, which further comprises at least one additional oligosaccharide.

11. The method of claim 1, wherein the 2′-fucosyllactose compound is administered to the subject orally.

12. The method of claim 9, wherein the composition is a pharmaceutical composition or a dietary supplement.

13. The method of claim 1, wherein the IBD is Crohn's disease.

14. The method of claim 1, wherein the IBD is ulcerative colitis.

15. The method of claim 1, wherein the subject has a daily fiber intake of less than 7 g/1000 kcal.

16. The method of claim 1, wherein the subject has a daily fiber intake of equal to or more than 7 g/1000 kcal.

17. The method of claim 1, wherein the subject is not receiving a corticosteroid.

18. The method of, wherein the subject is a FUT2 secretor.

19. The method of claim 1, wherein the subject is a FUT2 non-secretor.

20. The method of claim 1, wherein the subject is an adult.

21. The method of claim 1, wherein the subject is a child.

22. The method of claim 1, wherein the effective amount of the 2′-fucosyllactose compound is an amount equivalent to 1 mg/day to 20 mg/day of 2′-fucosyllactose, preferably equivalent to 1 mg/day to 15 mg/day of 2′-fucosyllactose, or more preferably equivalent to 1 mg/day to 10 mg/day of 2′-fucosyllactose.

23. The method of claim 1, wherein the 2′-fucosyllactose compound is 2′-fucosyllactose.

24. A method for treating inflammatory bowel disease (IBD), the method comprising administering to a subject in need thereof a 2′-fucosyllactose compound in an amount equivalent to 1 mg/day to 20 mg/day of 2′-fucosyllactose.

25. The method of claim 24, wherein the 2′-fucosyllactose compound is administered to the subject in an amount equivalent to 1 mg/day to 15 mg/day of 2′-fucosyllactose.

26. The method of claim 24, wherein the 2′-fucosyllactose compound is administered to the subject in an amount equivalent to 1 mg/day to 10 mg/day of 2′-fucosyllactose.

27. The method of claim 24, wherein the subject is a human patient at risk of developing IBD, suspected of having IBD, or having IBD.
